(19) United States
(12) Patent Application Publication
(10) Pub. No.: US 2023/0190735 A1
(43) Pub. Date: Jun. 22, 2023

(54) COMPOSITIONS COMPRISING VORICONAZOLE INHALATION POWDER AND METHODS OF MANUFACTURE AND USE THEREOF

(71) Applicant: TFF Pharmaceuticals, Inc., Austin, TX (US)

(72) Inventors: Dale J. Christensen, Cary, NC (US); John Koleng, Austin, TX (US)

(21) Appl. No.: 18/082,963

(22) Filed: Dec. 16, 2022

Related U.S. Application Data
(60) Provisional application No. 63/291,055, filed on Dec. 17, 2021.

(51) Int. Cl. A61K 31/496; A61K 9/00; A61P 31/10

(52) U.S. Cl. CPC A61K 31/496; A61K 9/0073; A61P 31/10

(57) ABSTRACT

The invention generally encompasses compositions and methods including inhalable voriconazole or a pharmaceutically acceptable salt thereof manufactured in amorphous form using thin film freezing. In various embodiments, the invention includes a dry powder voriconazole formulation that can be inhaled, for example, using a dry powder inhaler, and is well tolerated as a daily regimen for treating or preventing fungal infections. The compositions and methods of the invention avoid or reduce the systemic circulation of voriconazole and accordingly overcome the adverse events associated circulating voriconazole.

### CROSS-REFERENCE TO RELATED APPLICATIONS

This application claims the benefit of and priority to U.S. provisional patent application No. 63/291,055, which was filed on Dec. 17, 2021, and is incorporated herein by reference in its entirety.

### FIELD OF THE INVENTION

The invention generally encompasses compositions and methods including inhalable voriconazole or a pharmaceutically acceptable salt thereof manufactured in nanocrystalline form using thin film freezing. In various embodiments, the invention includes a dry powder voriconazole formulation that can be inhaled, for example, using a dry powder inhaler, and is well tolerated as a daily regimen for treating or preventing fungal infections. The compositions and methods of the invention avoid or reduce the systemic circulation of voriconazole and accordingly overcome the adverse events associated systemically circulating voriconazole.

### BACKGROUND OF THE INVENTION

Aspergillus is a fungus, which is commonly found in the soil, food, plant debris, and indoor environment. The spores are easily aerosolized and inhaled. In the respiratory mucosa, the spores may germinate into hyphae, which in turn can invade the mucosa leading to invasive pulmonary Aspergillosis (IPA). Both innate immune responses and inflammatory cells typically limit fungal growth and prevent disease in the majority of individuals. However, for immunocompromised or otherwise susceptible individuals, inhalation of spores can initiate a disease cascade that can lead to invasive pulmonary Aspergillosis (IPA) and invasive Aspergillosis (IA) that can be a severe, and often lethal, infection. The major risk factor for IPA is immunodeficiency brought on by multiple factors such as neutropenia, hematopoietic stem cell transplant (HSCT), solid organ transplantation, prolonged therapy with high-dose corticosteroids, hematological malignancy, cytotoxic therapy, advanced acquired immune deficiency syndrome (AIDS), chronic granulomatous disease and patients with liver failure. Moreover, increasing numbers of reports document IPA in immunocompromised patients who lack classic risk factors including those with severe chronic obstructive pulmonary disorder (COPD) and critically ill patients. In addition, patients with COPD have increased susceptibility to IPA, which can lead to increased morbidity. The increased risk is due to numerous factors. More recently, IPA was associated with severe influenza and SARS-CoV-2 infections.

Invasive Pulmonary Aspergillosis is also becoming an concerning infectious disease in intensive care unit (ICU) patients without the classical risk factors (neutropenia, leukemia, HSCT). In one retrospective study in a medical ICU, an IA incidence of 5.8% was found, with pulmonary involvement in most cases. Seventy percent (70%) of the cases were found in patients without leukemia or cancer, and the disease had a mortality rate exceeding 90%.

Voriconazole is a potent, broad spectrum triazole anti-fungal agent, where its primary mode of action is inhibition of fungal cytochrome P450-dependent 14-α-sterol demethylase CYP51, by binding to its heme group, an essential enzyme in ergosterol biosynthesis. Current treatment guidelines for Aspergillosis recommend oral and/or IV voriconazole (VFEND®) as first line therapy for IA. Increasingly, voriconazole is used in patients following bone marrow or solid organ transplant as prophylaxis for IPA, although only posaconazole has been approved for this indication. In the hospital setting, patients are typically treated initially with IV voriconazole if the patient is unable to take medications orally. The treatment is switched to oral voriconazole (e.g., 200 mg taken twice daily) as soon as the patient is stable and tolerates oral dosing.

However, with oral voriconazole, there is considerable variability in the systemic exposure due to the drug metabolism of voriconazole by CYP2C9, CYP2C19 and CYP3A4, and the reduction in absorption due to food (e.g., VFEND label). In addition to variability in metabolic clearance are the clinical drug-drug interactions (DDI) due to voriconazole's ability to inhibit CYP2B6, CYP2C9, CYP2C19 and CYP3A (e.g., VFEND label), thus challenging clinicians to prescribe a dose that will deliver neither too little nor too much to achieve the desired anti-fungal activity while minimizing potentially serious systemic side effects particularly in patients on immunosuppressive therapy that is metabolized by CYP3A, such as tacrolimus, leading to highly variable and unpredictable increases in tacrolimus systemic exposure.

In addition to the challenges of voriconazole metabolism and DDI for patients receiving oral or IV therapy, the amount of voriconazole that reaches the lung, the organ where the Aspergillosis infection is localized prior to spreading to distal organs, is a fraction of the total voriconazole administered to a patient. Notably, the voriconazole concentration that must be achieved in the lung for efficacy has not been determined. However, it was reported that epithelial lining fluid (ELF) concentrations 12 hours after the final dose of a 3-day twice daily (BID) dosing regimen with 200 mg of oral voriconazole averaged 8,827 ng/mL (range 4,369-35,172 ng/mL), while the corresponding plasma levels were 1,224 ng/mL (range 535-2,341 ng/mL). Estimates of ELF volumes range from 10-50 mL in the average human lung.

Given the potential to increase the local concentration of voriconazole while reducing drug-drug interactions and toxicity, the current inventors have developed voriconazole as a dry powder inhalation product to improve the delivery of voriconazole for potential treatment and/or prophylaxis in patients, for example, patients with or at high risk for IPA. The inventors have surprisingly discovered that voriconazole as a dry powder inhalation product improves the reliability and consistency of dosing of voriconazole, enhances the drug delivery to the target organ in IPA (i.e., the lungs), while reducing the systemic exposure that can lead to dose-limiting side effects. The inventors have further found that inhaling voriconazole directly bypasses the variability associated with gastrointestinal absorption and metabolism in patients on a variety of concomitant medications, which translates to a more reliable and universal dosing paradigm for treatment or prophylaxis.

### SUMMARY OF THE INVENTION

In various embodiments, the invention encompasses compositions and methods for treating or preventing a fungal infection in a subject in need thereof, the method comprising administering by inhalation to said subject voriconazole or a pharmaceutically acceptable salt thereof in an amount of about 10 mg to about 80 mg, wherein the administration by inhalation achieves a maximum circulating plasma concentration of voriconazole of less than 1,000 ng/mL.

In certain embodiments, the amount of voriconazole or a pharmaceutically acceptable salt thereof is about 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or about 80 mg.

In various embodiments, the amount of voriconazole or a salt thereof administered to a subject is about 5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or about 200 mg. In certain embodiments, the amount of voriconazole is administered in multiple unit dosage, for example, 8 administrations of 10 mg capsules using a dry powder inhaler.

In certain embodiments, the administration of voriconazole or a salt thereof comprises an amount of about 10 mg to about 120 mg, preferably about 40 mg to about 80 mg, most preferably about 40 mg or about 80 mg. The administration of voriconazole or a salt thereof is about once per day, twice per day, three times per day, four times per day, five times per day, six times per day, seven times per day, eight times per day, nine times per day, or ten times per day.

In various embodiments, the maximum circulating plasma concentration of voriconazole is less than about 1000 ng/mL, 900 ng/mL, 800 ng/mL, 700 ng/mL, 600 ng/mL, 500 ng/mL, 400 ng/mL, 300 ng/mL, 200 ng/mL, or less than 100 ng/mL.

In certain embodiments, the maximum circulating plasma concentration of voriconazole is less than 500 ng/mL. In certain embodiments, the maximum circulating plasma concentration of voriconazole is less than 300 ng/mL.

In certain embodiments, the fungal infection is caused by Aspergillus fumigatus, Aspergillus flavus, Aspergillus niger, Aspergillus terreus, Candida albicans, Candida glabrata, Candida krusei, Candida parapsilosis, Candida tropicalis, Fusarium spp., Fusarium solani, Scedosporium apiospermum, Candida lusitaniae, or Candida guilliermondii.

In certain embodiments, the fungal infection is invasive Aspergillosis, Candidemia, or esophageal Candidiasis.

In certain embodiments, the subject is suffering from a reactive airway disorder. In certain embodiments, the reactive airway disorder is asthma, chronic obstructive pulmonary disease (COPD), or bronchial infections.

In certain embodiments, the subject is immunodeficient. In certain embodiments, the immunodeficiency is caused by factors including neutropenia, hematopoietic stem cell transplant (HSCT), solid organ transplantation, prolonged therapy with high-dose corticosteroids, hematological malignancy, cytotoxic therapy, advanced acquired immune deficiency syndrome (AIDS), chronic granulomatous disease, and patients with liver failure.

In certain embodiments, the administration is at least once per day. In other embodiments, the administration is at least twice per day.

In certain embodiments, the voriconazole or a salt thereof is voriconazole inhalation powder.

In certain embodiments, the invention encompasses the invention encompasses compositions and methods for increasing delivery of voriconazole to the lung of a subject in need thereof, the method comprising administering to said subject by inhalation voriconazole or a salt thereof while minimizing systemic absorption and reducing drug-drug interactions. In certain embodiments, the reduction in drug-drug interactions is with CYP3A inhibitors. In certain embodiments, the methods comprising administering to said subject by inhalation voriconazole or a salt thereof reduce or avoid drug interactions in subjects concurrently administered drugs that are metabolized by cytochrome CYP3a enzymes. Accordingly, in certain embodiments, the administration to a subject by inhalation a therapeutic amount of voriconazole or a salt thereof minimizes or overcomes drug-drug interactions associated with administration of voriconazole orally or systemically (e.g., by IV injection).

In other embodiments, the invention encompasses compositions and methods for increasing delivery of voriconazole to the lung of a subject in need thereof, the method comprising administering to said subject by inhalation voriconazole or a salt thereof in an amount of about 10 mg to about 200 mg, preferably about 20 mg to about 80 mg, wherein the delivery of voriconazole to the lung by inhalation results in about 50-fold higher relative concentration in the lung compared to the oral administration of voriconazole after about 12 hours following administration.

In various embodiments, the amount of voriconazole or a salt thereof administered to a subject is about 1 mg, 2 mg, 4 mg, 6 mg, 8 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or about 200 mg.

In various embodiments, the amount of voriconazole or a salt thereof is administered to a subject in a dosage form, for example, a capsule including voriconazole or a salt thereof in an amount of about 1 mg, 2 mg, 4 mg, 6 mg, 8 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or about 200 mg. In certain embodiments, the dosage form is administered multiple times, for example, about once per day, twice per day, three times per day, four times per day, five times per day, six times per day, seven times per day, eight times per day, nine times per day, or ten times per day.

In certain embodiments, the administration of voriconazole or a salt thereof comprises an amount of about 10 mg to about 120 mg, preferably about 40 mg to about 80 mg, most preferably about 40 mg or about 80 mg. The administration of voriconazole or a salt thereof is about once per day, twice per day, three times per day, four times per day, or five times per day.

In various embodiments, the delivery of voriconazole to the lung by inhalation results in about 10-fold to about 200-fold higher, preferable about 10-fold, 20-fold, 30-fold, 40-fold, 50-fold, 60-fold, 80-fold, 90-fold, 100-fold, 150-fold, 200-fold, most preferably about 50-fold, higher relative concentration in the lung compared to the oral administration of voriconazole after about 12 hours following administration. In various embodiments, the higher relative concentration in the lung compared to the oral administration of voriconazole is after about 30 minutes, 1 hour, 2 hours, 3 hours, 4 hours, 5 hours, 7 hours, 8 hours, 9 hours, 10 hours, 11 hours, or after about 12 hours following administration.

In certain embodiments, the delivery of voriconazole to the lung results in about 100-fold higher relative concentration in the lung compared to the oral administration of voriconazole.

In certain embodiments, the delivery of voriconazole to the lung results in about 200-fold higher relative concentration in the lung compared to the oral administration of voriconazole.

In certain embodiments, the subject is suffering from a fungal infection caused by Aspergillus fumigatus, Aspergillus flavus, Aspergillus niger, Aspergillus terreus, Candida albicans, Candida glabrata, Candida krusei, Candida parapsilosis, Candida tropicalis, Fusarium spp., Fusarium solani, Scedosporium apiospermum, Candida lusitaniae, or Candida guilliermondii.

In certain embodiments, the subject is suffering from a fungal infection caused by Invasive Aspergillosis, Candidemia, or esophageal Candidiasis.

In certain embodiments, the subject is immunodeficient.

In certain embodiments, the immunodeficiency is caused by factors comprising neutropenia, hematopoietic stem cell transplant (HSCT), solid organ transplantation, prolonged therapy with high-dose corticosteroids, hematological malignancy, cytotoxic therapy, advanced acquired immune deficiency syndrome (AIDS), chronic granulomatous disease, and patients with liver failure.

In certain embodiments, the administration is at least once per day. In other embodiments, the administration is at least twice per day.

In certain embodiments, the voriconazole or a salt thereof is voriconazole inhalation powder.

In certain embodiments, the administration of voriconazole or a salt thereof comprises an amount of about 10 mg to about 120 mg. In certain embodiments, the administration of voriconazole or a salt thereof comprises an amount of about 40 mg to about 80 mg. In certain embodiments, the administration of voriconazole or a salt thereof comprises an amount of about 80 mg.

In certain embodiments, the voriconazole or a salt thereof is voriconazole inhalation powder.

In other embodiments, the invention encompasses methods and compositions for reducing systemic circulation of voriconazole compared with the oral administration of voriconazole comprising administering to said subject by inhalation voriconazole or a salt thereof in an amount of about 20 mg to about 80 mg, wherein the administration by inhalation achieves a maximum circulating plasma concentration of voriconazole of less than 1,000 ng/mL.

In certain embodiments, the maximum circulating plasma concentration of voriconazole is less than 500 ng/mL. In certain embodiments, the maximum circulating plasma concentration of voriconazole is less than 300 ng/mL.

In various embodiments, the amount of voriconazole or a salt thereof administered to a subject is about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or about 200 mg. In certain embodiments, the amount of voriconazole or a pharmaceutically acceptable salt thereof is about 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or about 80 mg.

In certain embodiments, the administration of voriconazole or a salt thereof comprises an amount of about 10 mg to about 120 mg, preferably about 40 mg to about 80 mg, most preferably about 40 mg or about 80 mg. The administration of voriconazole or a salt thereof is about once per day, twice per day, three times per day, four times per day, or five times per day.

In certain embodiments, the voriconazole or a salt thereof is voriconazole inhalation powder.

In various embodiments, the maximum circulating plasma concentration of voriconazole is less than about 1000 ng/mL, 900 ng/mL, 800 ng/mL, 700 ng/mL, 600 ng/mL, 500 ng/mL, 400 ng/mL, 300 ng/mL, 200 ng/mL, or less than 100 ng/mL.

In certain embodiments, the maximum circulating plasma concentration of voriconazole is less than 500 ng/mL. In certain embodiments, the maximum circulating plasma concentration of voriconazole is less than 300 ng/mL.

In certain embodiments, the fungal infection is caused by Aspergillus fumigatus, Aspergillus flavus, Aspergillus niger, Aspergillus terreus, Candida albicans, Candida glabrata, Candida krusei, Candida parapsilosis, Candida tropicalis, Fusarium spp., Fusarium solani, Scedosporium apiospermum, Candida lusitaniae, or Candida guilliermondii.

In certain embodiments, the fungal infection is Invasive Aspergillosis, Candidemia, or esophageal Candidiasis.

In certain embodiments, the subject is immunodeficient.

In certain embodiments, the immunodeficiency is caused by factors comprising neutropenia, hematopoietic stem cell transplant (HSCT), solid organ transplantation, prolonged therapy with high-dose corticosteroids, hematological malignancy, cytotoxic therapy, advanced acquired immune deficiency syndrome (AIDS), chronic granulomatous disease, and patients with liver failure.

In certain embodiments, the administration is at least once per day. In certain embodiments, the administration is at least twice per day.

In certain embodiments, the voriconazole or a salt thereof is voriconazole inhalation powder.

In certain embodiments, the administration of voriconazole or a salt thereof comprises an amount of about 10 mg to about 120 mg. In certain embodiments, the administration of voriconazole or a salt thereof comprises an amount of about 40 mg to about 80 mg. In certain embodiments, the administration of voriconazole or a salt thereof comprises an amount of about 80 mg.

In certain embodiments, the voriconazole or a salt thereof is voriconazole inhalation powder.

### BRIEF DESCRIPTION OF THE DRAWINGS

FIG. 1 illustrates a theoretical depiction of drug deposition of voriconazole following oral administration (left side, 200 mg) and inhaled administration (right side, 40-80 mg). The illustration shows that inhaled administration of voriconazole deposits greater percentage of drug in the lungs, with lower gastrointestinal and systemic exposure compared to the oral route of administration.

FIG. 2A illustrates Voriconazole Plasma Concentration Time Plots for all SAD and MAD subjects; Semilog after a Single Inhaled Dose (SAD and MAD Cohorts Combined). Voriconazole (Mean+SD) Concentration Time Plot After a Single Inhalation. FIG. 2B illustrates Voriconazole Plasma Concentration Time Plots for Extensive Metabolizers only; Semilog.

FIG. 3 illustrates Voriconazole Plasma Concentration Time Plots for CYP2C19 Extensive and Poor Metabolizers. Voriconazole (Individual or Mean[+SD]) Plasma Concentration Time Plot After a Single Inhalation of VIP Doses on Day 1 that included Extensive Metabolizers (EM) and Poor Metabolizers (PM); SAD and MAD Cohorts Combined.

FIG. 4 illustrates Voriconazole Plasma Concentration Time Plot of 40 mg and 80 mg on Day 1 and Day 7. Voriconazole (Mean+SD) Plasma Concentration Time Plot Dose 1 (Day 1) and Dose 13 (Day 7) Overlay for 40 mg and 80 mg (Extensive Metabolizers only; Semilog).

FIG. 5 illustrates Log-Log Plots of Dose vs PK Parameters in All Subjects (FIGS. 5A & 5B: Panels A) and Extensive Metabolizers Only (FIGS. 5A & 5B: Panels B). FIG. 5A illustrates Cmax and FIG. 5B illustrates AUCtau. Log-Log Plots of Dose vs PK Parameters of Voriconazole on Day 1 and Dose 13 (Day 7) for Cmax (FIG. 5A) and AUCtau (FIG. 5B). Panels A include all subjects and Panels B include Extensive Metabolizers Only.

FIG. 6 illustrates characterization of voriconazole/mannitol (95:5 w/w) mixture by XRPD indicated a crystalline solid.

FIG. 7 illustrates SEM-EDX of a 50:50 mixture confirmed the suitability of the thin-film freezing (TFF) process with the voriconazole phase-separated from the mannitol.

FIG. 8 illustrates an exemplary schematic of a manufacturing protocol.

FIG. 9 illustrates Mean (+SD) Voriconazole Plasma Concentrations vs. Time by Dose Levels, SAD—All Subjects (Log-linear Axes).

FIG. 10 illustrate Mean (+SD) Voriconazole Plasma Concentrations vs. Time by Dose Levels, SAD—CYP2C19 Extensive Metabolizers Only (Log-linear Axes).

FIG. 11 illustrates Mean (±SD) Voriconazole Concentrations after the First and Last Doses from MAD, All Dose Levels, All Subjects.

FIG. 12 illustrates Mean (±SD) Voriconazole Concentrations After the First and Last Doses from MAD, All Dose Levels: Extensive Metabolizers Only.

FIG. 13 illustrates Voriconazole Mean Systemic Exposure after 40 mg and 80 mg Single Dose Voriconazole Inhalation Powder in TFF-V1-001 and TFF-V1-002.

FIG. 14 illustrates Voriconazole Mean Systemic Exposure after 40 mg and 80 mg Repeat Dose Voriconazole Inhalation Powder in TFF-V1-001 and TFF-V1-002.

FIG. 15 illustrates that tacrolimus levels/doses remained unchanged after administration of inhaled voriconazole supporting the concept of reduced drug-drug interactions using inhalable voriconazole.

FIG. 16 illustrates the stabilization of lung function after administration of inhalable voriconazole.

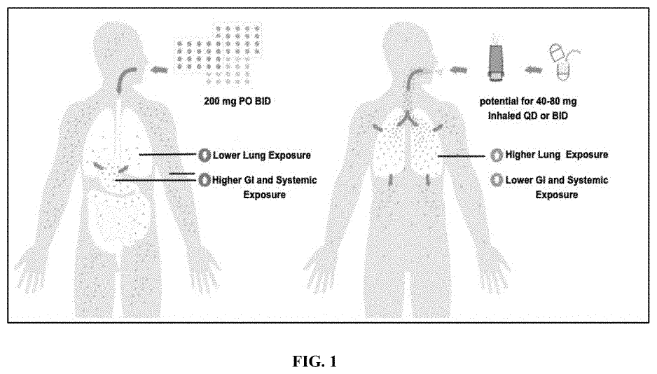

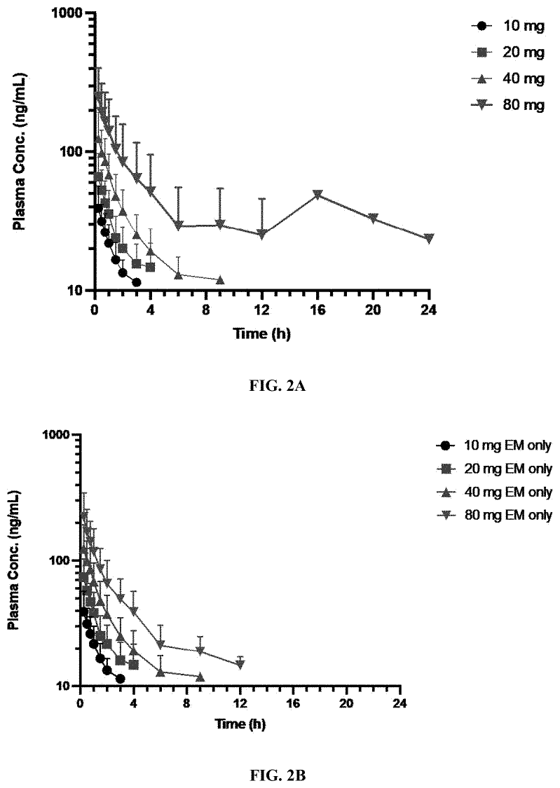

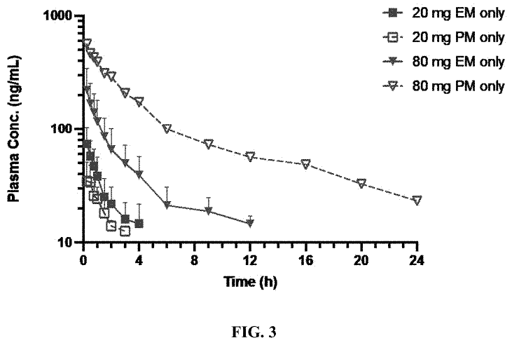

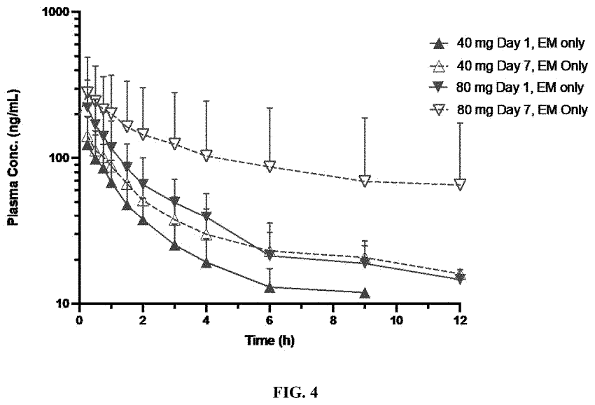

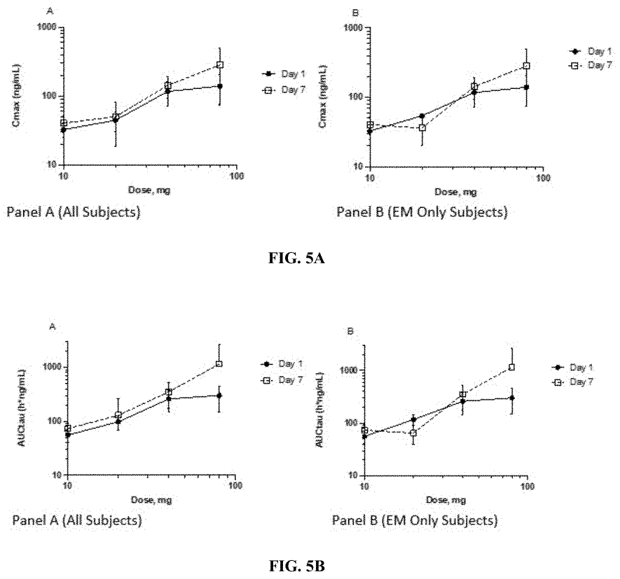

### DETAILED DESCRIPTION OF THE INVENTION

### Definitions

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

“About” when referring to a value includes the stated value+/−10% of the stated value. For example, about 50% includes a range of from 45% to 55%, while about 20 molar equivalents includes a range of from 18 to 22 molar equivalents. Accordingly, when referring to a range, “about” refers to each of the stated values+/−10% of the stated value of each end of the range. For instance, a ratio of from about 1 to about 3 (weight/weight) includes a range of from 0.9 to 3.3.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats). The term “mammal” refers to any mammalian species such as a human, mouse, rat, dog, cat, hamster, guinea pig, rabbit, livestock, and the like.

Provided are also pharmaceutically acceptable salts and tautomeric forms of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” refers to salts that retain the biological effectiveness and properties of voriconazole and which are not biologically or otherwise undesirable. “Pharmaceutically acceptable salts” or “physiologically acceptable salts” include, for example, salts with inorganic acids and salts with an organic acid. Those skilled in the art will recognize various synthetic methodologies that may be used to prepare nontoxic pharmaceutically acceptable addition salts. Pharmaceutically acceptable acid addition salts may be prepared from non-toxic inorganic and organic acids. The pharmaceutically acceptable salts can be synthesized from the parent compound which contains a basic or acidic moiety by conventional chemical methods. Lists of suitable salts are found in Remington's Pharmaceutical Sciences, 17^{th} Ed., (Mack Publishing Company, Easton, 1985), p. 1418, Berge et at, J. Pharm. Sci., 1977, 66(1), 1-19 and in Stahl et al., Handbook of Pharmaceutical Salts: Properties, Selection, and Use, (Wiley, 2002). Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21^{st} Edition, Lippincott Williams and Wilkins, Philadelphia, Pa., 2006.

Voriconazole or its pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). It is understood that the aspect encompasses, but is not limited to, the specific diastereomerically or enantiomerically enriched form. Where chirality is not specified but is present, it is understood that the aspect is directed to either the specific diastereomerically or enantiomerically enriched form; or a racemic or scalemic mixture of such compound(s). As used herein, “scalemic mixture” is a mixture of stereoisomers at a ratio other than 1:1.

“Racemates” refers to a mixture of enantiomers. The mixture can include equal or unequal amounts of each enantiomer.

“Stereoisomer” and “stereoisomers” refer to a difference in the chirality of one or more stereocenters. Stereoisomers include enantiomers and diastereomers. Voriconazole may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the ark (see, e.g., Chapter 4 of Advanced Organic Chemistry, 4th ed., J. March, John Wiley and Sons, New York, 1992).

The invention generally encompasses compositions and methods including voriconazole or a pharmaceutically acceptable salt thereof manufactured in nanocrystalline form using thin film freezing. In various embodiments, the invention includes a dry powder voriconazole formulation referred to herein as “Voriconazole Inhalation Powder” that can be inhaled, for example, using a dry powder inhaler, and is well tolerated as a daily regimen, preferably twice daily.

In various embodiments, the inventors found that the claimed compositions and methods including Voriconazole Inhalation Powder at doses up to, for example, 80 mg twice a day delivered low plasma exposure (e.g., Cₘₐₓ, AUC) relative to the recommended oral dose (i.e., 200 mg BID), which provides a treatment benefit in patients.

In various embodiments, the compositions and methods including Voriconazole Inhalation Powder can act as a therapeutic in the treatment of invasive fungal infections, for example, in critical patient populations in order to overcome extensive drug-drug interactions (DDI) with oral or intravenous administration of voriconazole.

Voriconazole's DDI potential is due to it being an inhibitor of four of the CYP 450 enzymes that are involved in metabolism of approximately 67% of the drugs currently marketed (e.g., CYP2B6, CYP2C9, CYP2C19 and CYP3A). Table 1 illustrates the literature values for Ki and two oral Cₘₐₓ values as well as the Cₘₐₓ value observed in this study for the Voriconazole Inhalation Powder at 80 mg dose.

TABLE 1
Voriconazole Reversible Inhibition R₁ Values from Oral
or Inhaled Routes of Administration

| | | Imax | Imax | | | Imax | R₁ | R_{1,gut}⁶ |
|---|---|---|---|---|---|---|---|---|
| | Ki² | oral³ | oral⁴ | R₁ oral | R_{1,gut} | inhaled⁵ | In- | In- |
| CYP¹ | (μM) | (μM) | (μM) | (Range) | oral | (μM) | haled | haled |
| 2B6 | 0.5 | 6.61 | 3.50 | 8.01-14.2 | 4580 | 0.925 | 2.85 | 19.3 |
| 2C19 | 5.1 | | | 1.69 = 2.30 | 450 | | 1.18 | 2.80 |
| 2C9 | 2.79 | | | 2.56-3.37 | 822 | | 1.33 | 4.28 |
| 3A | 0.66 | | | 6.31-11.0 | 3470 | | 2.40 | 14.9 |

Equations for Basic Models of Reversible Inhibition
R₁ = 1 + (I_{max,u}/K_{i,u})
R_{1,gut} = 1 + (I_{gut}/K_{i,u})
Imax,u is the maximal unbound plasma concentration of the interacting drug at steady state
Imax = Cₘₐₓ (ng/mL)/MW of voriconazole of 349.32
I_{gut} is the intestinal luminal concentration of the interacting drug calculated as the dose/250 mL
Ki,u is the unbound inhibition constant determined in vitro
If R₁ ≥ 1.02 or the R_{1,gut} ≥ 11 then a clinical DDI study with a sensitive index substrate may be required.
¹calculations are based on total drug instead of unbound Ki and Imax.
²Ki values based on Jeong 2009
³Oral Imax based on 200 mg q12h from VFEND label
⁴oral Imax based on Cₘₐₓ from Anderson 2017.
⁵based on Cₘₐₓ from 80 mg VIP on Day 7 (13^{th} dose)
⁶assumption of 1% of dose is delivered to the stomach

The CYP Ki are CYP2B6 (Ki<0.5 μM), CYP2C9 (Ki=2.79 μM), CYP2C19 (Ki=5.1 μM) and CYP3A (Ki=0.66 μM), and PK data are illustrated in Table 2.

TABLE 2
Geometric Mean (% CV) Plasma Voriconazole PK Parameters in
Adults Receiving Oral Voriconazole Vs. Inhaled Voriconazole

| | Oral Voriconazole^{a} | | | |
|---|---|---|---|---|
| | | 200 mg | 300 mg | Voriconazole |
| | 400 mg Oral | Oral | Oral | Inhalation Powder^{b} |
| | (loading dose) | q 12 h | q 12 h | 80 mg |
| N | 17 | 48 | 16 | 6 |
| AUC₁₂ | 9.31 (38) | 12.4 (78) | 34.0 (53) | 1.150 (1.550) |
| (μg · h/mL) | | | | |
| Cₘₐₓ | 2.30 (19) | 2.31 (48) | 4.74 (35) | 0.283 (0.207) |
| (μg/mL) | | | | |
| Cₘᵢₙ | — | 0.46 (120) | 1.63 (79) | 0.0436 (0.0902) |
| (μg/mL) | | | | |

^{a}Oral voriconazole parameters were estimated based on non-compartmental analysis from 5 pharmacokinetic studies (Source VFEND package insert).
^{b}Inhaled voriconazole parameters were taken from MAD part after the 13^{th} dose.
Abbreviations: AUC₁₂ = area under the curve over 12 hour dosing internal; Cₘₐₓ = maximum plasma concentration; Cₘᵢₙ = Minimum plasma concentration; CV = coefficient of variation; MAD = Multiple ascending dose.

Tables 3 and 4 illustrate pharmacokinetic parameters after administration of a single dose and multiple doses, respectively.

TABLE 3
Voriconazole PK Parameters after a Single Inhaled Dose (SAD and MAD Cohorts Combined)

| | Voriconazole | | Voriconazole | | Voriconazole | | Voriconazole | |
|---|---|---|---|---|---|---|---|---|
| Parameter | Inhalation Powder | | Inhalation Powder | | Inhalation Powder | | Inhalation Powder | |
| (units) ^{a} | 10 mg | | 20 mg | | 40 mg | | 80 mg | |
| Tₘₐₓ (h)^{b} | 10 | 0.25 (0.25, 1.05) | 8 | 0.32 (0.25,0.52) | 12 | 0.25 (0.25, 0.27) | 11 | 0.25 (0.25, 0.53) |
| | | | | [0.38]^{e} | | | | [0.25] ^{e} |
| Tₗₐₛₜ (h)^{b} | 10 | 2.00 (1.00, 3.00) | 8 | 3.00 (1.50, 3.00) | 12 | 4.00 (2.00, 9.00) | 11 | 6.00 (2.00, 12.00) |
| | | | | [2.47]^{e} | | | | [24.0] ^{e} |
| Cₘₐₓ^{c} | 10 | 40.7 (35.9) | 8 | 71.8 (41.9) | 12 | 108 (62.2) | 11 | 191 (71.8) |
| (ng/mL) | | | | [35.1]^{e} | | | | [563] ^{e} |
| AUCₜₐᵤ^{c} | 9 | 62.7 (44.6) | 8 | 108 (58.5) | 12 | 205 (60.9) | 11 | 377 (75.6) |
| (h*ng/mL) | | | | [79.0] ^{e} | | | | [1890] ^{e} |
| AUC_{inf}^{c} | 9 | 62.3 (44.3) | 8 | 108 (60.0) | 12 | 207 (21.9) | 11 | 399 (80.7) |
| (h*ng/mL) | | | | [78.0] ^{e} | | | | [2620] ^{e} |
| t 1/2 (h)^{d} | 9 | 1.20 (0.393) | 8 | 1.41 (0.603) | 12 | 1.80 (0.506) | 11 | 2.86 (1.85) |
| | | | | [1.56] ^{e} | | | | [7.54] ^{e} |

N = Number of observations
Abbreviations:
AUC_{inf} = Area under the concentration-time curve from time 0 extrapolated to infinity; AUCₜₐᵤ = Area under the plasma concentration-time curve over the dosing interval; Cₘₐₓ-maximum concentration; t½ = half life; Tₗₐₛₜ = Time of last observed concentration; Tₘₐₓ = time to maximum concentration.
^{a} PK parameters with summary statistics are of extensive metabolizers for CYP2C19. Data for individual(s) identified as poor metabolizers within a treatment group are presented in [brackets].
^{b}Values are median (min, max)
^{c}Values represent geometric mean and (% CV)
^{d}values are arithmetic mean (SD)
^{e} [median value of poor metabolizer for 20 mg and value of 80 mg that had PK parameters]

TABLE 4
Voriconazole PK Parameters on Day 7 after Dose 13 of BID Administration

| | Voriconazole | | Voriconazole | | Voriconazole | | Voriconazole | |
|---|---|---|---|---|---|---|---|---|
| Parameter | Inhalation Powder | | Inhalation Powder | | Inhalation Powder | | Inhalation Powder | |
| (units) ^{a} | 10 mg | | 20 mg | | 40 mg | | 80 mg | |
| Cₘₐₓ^{c} | 6 | 39.3 (27.1) | 3 | 33.9 (46.7) | 6 | 134 (44.1) | 6 | 227 (87.2) |
| (ng/mL) | | | | [93.5]^{e} | | | | |
| AUCₜₐᵤ^{c} | 6 | 71.0 (30.5) | 3 | 61.8 (39.8) | 6 | 315 (57.4) | 6 | 664 (153) |
| (h*ng/mL) | | | | [322] ^{e} | | | | |
| Cₘᵢₙ^{c} | 6 | 1.67 (4.08) | 3 | 0.00 (0.00) | 6 | 2.67 (6.53) | 6 | 43.6 (90.2) |
| (ng/mL) | | | | [0.00] ^{e} | | | | |
| Tₘₐₓ (h)^{b} | 6 | 0.25 (0.25, 0.28) | 3 | 0.25 (0.25, 0.50) | 6 | 0.25 (0.25, 0.50) | 6 | 0.25 (0.25, 0.50) |
| | | | | [0.78] ^{e} | | | | |
| t½ (h)^{d} | 6 | 1.71 (0.767) | 3 | 1.22 (0.148) | 6 | 2.66(1.20) | 6 | 5.56 (4.72) |
| | | | | [2.73] ^{e} | | | | |
| CLss/F^{c} | 6 | 141 (30.5) | 3 | 323 (39.8) | 6 | 127 (57.4) | 6 | 121 (153) |
| (L/h) | | | | [62.2] ^{e} | | | | |
| Vz/F^{c} (L) | 6 | 318 (31.6) | 3 | 566 (46.4) | 6 | 451 (30.6) | 6 | 779 (61.5) |
| | | | | [245] ^{e} | | | | |
| R_{acc} | 4 | 1.14 (24.9) | 2 | 0.643 (58.9) | 6 | 1.31 (26.1) | 6 | 2.57 (79.6) |
| AUC₀₋₁₂^{c} | | | | [3.43] ^{e} | | | | |
| R_{acc} Cₘₐₓ^{c} | 5 | 1.14 (16.8) | 2 | 0.772 (33.1) | 6 | 1.23 (21.7) | 6 | 1.81 (67.6) |
| | | | | [2.03] ^{e} | | | | |

Abbreviations:
AUC_{inf} = Area under the concentration-time curve from time 0 extrapolated to infinity; AUCₜₐᵤ = Area under the plasma concentration-time curve over the dosing interval; CLss/F = Apparent total body clearance after oral administration; Cₘₐₓ-maximum concentration, N = Number of observations; Racc AUC₀₋₁₂ = Accumulation ratio for AUC₀₋₁₂ₕ; R_{acc} Cₘₐₓ = Accumulation ratio for Cₘₐₓ; t½ = half-life; Tₗₐₛₜ = Time of last observed concentration; Tₘₐₓ = time to maximum concentration; Vz/F = Apparent volume of distribution during the terminal elimination phase after oral administration.
^{a} PK parameters with summary statistics are of extensive metabolizers for CYP2C19. Data for individual(s) identified as poor metabolizers within a treatment group are presented in [brackets].
^{b}Values are median (min, max)
^{c}Values represent geometric mean and (% CV)
^{d}values are arithmetic mean (SD)
^{e} [value of poor metabolizer for 20 mg

The invention further encompasses compositions and methods including Voriconazole Inhaled Powder that ameliorate or avoid drug-drug interactions (DDI). In certain embodiments, the inhalation delivery of Voriconazole Inhaled Powder does not impact the absorption and metabolism of drugs metabolized by CYP 450 enzymes within the GI epithelium, for example, tacrolimus, which is a critical immunosuppressant used after solid organ transplant and is metabolized by CYP3A5.

In other embodiments, the compositions and methods including Voriconazole Inhaled Powder including up to about an 80 mg dose avoids or reduces the potential for systemic/liver DDI based on metabolism by CYP 450 enzymes including, for example, CYP2C19 and CYP2C9.

In other embodiments, the compositions and methods including Voriconazole Inhaled Powder result in lower systemic concentrations of circulating voriconazole compared with oral administration. In certain embodiments, the compositions and methods including Voriconazole Inhaled Powder result in systemic exposure after an 80 mg inhaled dose that is lower than the therapeutic trough concentration in plasma, 1.7 μg/mL, after oral administration, required to ensure treatment success against invasive fungal infections. However, at this trough concentration there are significant clinical DDI, such as with tacrolimus, which is critical in ensuring no organ rejection occurs. Accordingly, the inventors have surprisingly found that the lower exposure systemically following administration of the Voriconazole Inhaled Powder after inhalation with the high concentrations in the lung provides a superior alternative to oral administration in cases of patients, for example, with lung transplants.

In other embodiments, the compositions and methods including Voriconazole Inhaled Powder include voriconazole at doses from about 10 mg to about 80 mg, for example, about 10, 20, 30, 40, 50, 60, 70 or about 80 mg of voriconazole or a pharmaceutically acceptable salt thereof.

In other embodiments, the compositions and methods including Voriconazole Inhaled Powder exhibited a dose proportional increases in exposure after single and repeated administration with the clearance of the drug being relatively consistent at steady state; C_{LSS/F} geometric mean values ranging from about 141 L/h to about 121 L/h for 10 mg and 80 mg, respectively. In various embodiments, the Cₘₐₓ of the 80 mg inhaled dose was about 227 ng/mL compared to an oral 200 mg dose of 2310 ng/mL.

In certain embodiments, the compositions and methods including Voriconazole Inhaled Powder provide less voriconazole reaching systemic circulation for a given mg dose by the inhaled route than by the oral route. However, this does not mean that inhaled voriconazole will not be effective against invasive Aspergillosis. In various embodiments, the compositions and methods including Voriconazole Inhaled Powder provide, given the deposition of 50% of the inhaled voriconazole and the alveolar surface volume in the lungs of 50 mL, a concentration on the airway surface available to the immediate lung tissue of about 1600 μg/mL (80,000 μg/50 mL), which is sufficient to treat all wild type and resistant variants of Aspergillus spp. Accordingly, the compositions and methods including Voriconazole Inhaled Powder result in lower exposure to systemic voriconazole by the inhaled route and accordingly allow for prophylaxis and treatment and improve the outcomes of lung transplant recipients.

In other embodiments, the compositions and methods including Voriconazole Inhaled Powder comprise longer t½ at doses of 40 mg and 80 mg result in a transient depot of voriconazole in the lungs that leads to a longer half-life due to continued absorption from this reserve of drug within the dosing interval.

In other embodiments, the compositions and methods including Voriconazole Inhaled Powder can be used as a primary prophylactic or a treatment option, for example, in immunocompromised individuals undergoing hematopoietic stem cell transplantation and those with lung transplants that avoids DDI associated with other therapeutics being administered.

In other embodiments, the compositions and methods including Voriconazole Inhaled Powder provide prophylaxis in immunocompromised individuals and improve the risk to benefit ratio with the use of a less systemically available agent, such as the compositions and methods including Voriconazole Inhaled Powder.

In various embodiments, Voriconazole Inhalation Powder can be used as an alternative to oral voriconazole treatment. In other embodiments, the compositions and methods including Voriconazole Inhaled Powder avoid or reduce the limitations related oral or intravenous administration of voriconazole including systemic side effects, multi-system toxicities, multiple potential DDIs, and, as for all antifungal agents, concern about antifungal resistance emergence following prolonged treatment.

In other embodiments, the compositions and methods including Voriconazole Inhaled Powder comprise delivery of voriconazole to the infected organ (e.g., the lungs) directly and convey benefits of treating infection in the lungs more effectively and reduce the progression to generalized invasive Aspergillosis.

### Formulations of Voriconazole Inhaled Powder

The compositions including Voriconazole Inhaled Powder comprise a finished drug product including a drug-device combination intended to deliver the formulated voriconazole drug product as a dry powder into the lungs via the oral inhalation route of administration. In certain embodiments, the formulated Voriconazole Inhalation Powder is supplied in a capsule, for example, a hard, two-piece HPMC capsules (e.g., Size 3) for administration using a commercially available dry powder inhaler (DPI) device. In certain embodiments, Voriconazole Inhalation Powder is available as 10 mg voriconazole capsules that can be directly inserted into a DPI. In certain embodiments, a capsule-based DPI device (e.g., RS00 Model 8 Monodose) manufactured by Plastiape S.p.A. (Osnago, Italy) is used for administration of Voriconazole Inhalation Powder. In various embodiments, to achieve a therapeutic level of voriconazole, one or more 10 mg doses can be administered, for example, one 10 mg dose, two 10 mg doses, three 10 mg doses, four 10 mg doses, five 10 mg doses, six 10 mg doses, seven 10 mg doses, eight 10 mg doses or more doses to achieve the appropriate amount of voriconazole.

In one embodiment, Voriconazole Inhalation Powder is comprised of 95:5% w/w voriconazole/mannitol in a capsule, for example, a hard, two-piece HPMC capsule (e.g., Size 3).

Table 5 below provides an illustrative embodiment of a Voriconazole Inhalation Powder formulation of the invention.

TABLE 5
Exemplary Voriconazole Inhalation Powder Formulation

| | Quality | Component | Composition | |
|---|---|---|---|---|
| Component | Standard | Function | Quantity (mg) | Percent (%) |
| Voriconazole | USP/Ph. Eur. | Drug substance | 10.00 | 95.0 |
| Mannitol | USP | Diluent | 0.53 | 5.0 |
| Acetonitrile¹ | High purity grade | Processing aid | N/A | N/A |
| Purified water¹ | USP/Ph. Eur. | Processing aid | N/A | N/A |
| TOTAL | N/A | N/A | 10.53 | 100 |
| Empty Hard HPMC | In-house² | Encapsulation | 1 each | N/A |
| Capsule, Size 3, White | | | | |

HPMC = Hypromellose; N/A = not applicable; Ph. Eur. = European Pharmacopeia; USP = United States Pharmacopeia
¹Removed during processing.
²The hard, two-piece HPMC capsule shell is composed of Hypromellose (USP/Ph. Eur.) and titanium dioxide (USP/Ph. Eur.). Although the capsule shell is not a compendial item, it is composed of compendial materials that are tested to the current compendium.

In another embodiment, the formulation of Voriconazole Inhalation Powder is comprised of TFF processed voriconazole and leucine, dry blended with magnesium stearate (93.1:4.9:2% w/w voriconazole/leucine/magnesium stearate) in a capsule (e.g., Size 3).

Table 6 below provides another illustrative embodiment of a Voriconazole Inhalation Powder formulation of the invention.

TABLE 6
Exemplary Voriconazole Inhalation Powder Composition

| | | Component | Composition | |
|---|---|---|---|---|
| Component | Quality Standard | Function | Quantity (mg) | Percent (%) |
| Voriconazole | USP/Ph. Eur. | Drug substance | 10.00 | 93.1 |
| Leucine | USP | Diluent | 0.53 | 4.9 |
| Magnesium stearate | NF/Ph. Eur. | Glidant | 0.21 | 2.0 |
| Acetonitrile¹ | High purity grade | Processing aid | N/A | N/A |
| Purified water¹ | USP/Ph. Eur. | Processing aid | N/A | N/A |
| TOTAL | N/A | N/A | 10.74 | 100 |
| Empty Hard HPMC | In-house² | Encapsulation | 1 each | N/A |
| Capsule, Size 3, White | | | | |

HPMC = Hypromellose; N/A = not applicable; Ph. Eur. = European Pharmacopeia; USP = United States Pharmacopeia
¹Removed during processing.
²The hard, two-piece HPMC capsule shell is composed of Hypromellose (USP/Ph. Eur.) and titanium dioxide. (USP/Ph. Eur.). Although the capsule shell is not a compendial item, it is composed of compendial materials that are tested to the current compendium.

Formulation screening was initially conducted on voriconazole with a variety of excipients (bulking agents, de-agglomerating agents and hydrophobic agents). Mixtures were aerosolized using the Plastiape RS01 (high resistance, low resistance and low resistance with longer mouthpiece) and were evaluated using a Malvern Spraytec Laser Diffraction (LD) analyzer. Aerodynamic particle size parameters were assessed. Table 7 summarizes the excipients that were evaluated in conjunction with voriconazole.

TABLE 7
Formulation Screening Excipients Evaluated
for Use with Voriconazole

| | | Quantities |
|---|---|---|
| Function | Excipient | Evaluated (w/w) |
| Bulking Agent | Lactose | 5% |
| | Mannitol | 5% |
| De-agglomeration Agent | Glycine | 2.5%, 5.0% |
| | Arginine | 1.0%, 2.5% |
| | Leucine | 1.0%, 2.5% |
| Hydrophobic Agent | Lecithin | 0.1%, 0.5%, 1.0% |
| | Magnesium stearate | 0.1%, 1.0%, 5.0% |
| | Calcium carbonate | 0.1%, 1.0% |
| | Titanium dioxide | 0.1%, 1.0% |

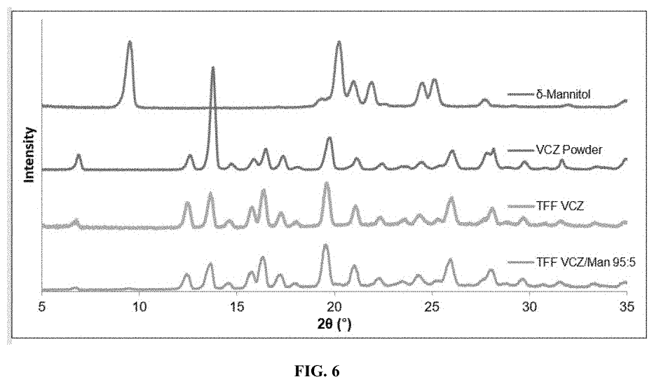

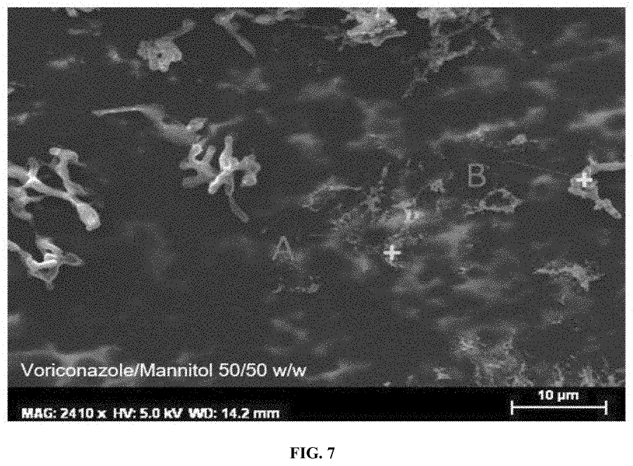

Characterization of voriconazole/mannitol (95:5 w/w) mixture by XRPD indicated a crystalline solid as illustrated in FIG. 6. SEM-EDX of a 50:50 mixture confirmed the suitability of the thin-film freezing (TFF) process with the voriconazole phase-separated from the mannitol (see FIG. 7).

The excipients and adjuvants that may be used in the present invention are generally defined for this application as compounds that enhance the efficiency and/or efficacy of the active agents. It is also possible to have more than one excipient, adjuvant, or even active agent in a given formulation. Non-limiting examples of additional agents that may be included in the solutions that are to be made in accordance with the present invention include: surfactants, fillers, stabilizers, polymers, protease inhibitors, antioxidants and absorption enhancers. Excipients may be selected and added to either the drug/organic mixture or to the aqueous solution, either before or after the drug particles are formed, in order to enable the drug particles to be homogeneously admixed for appropriate administration. Suitable excipients include polymers, absorption enhancers, solubility enhancing agents, dissolution rate enhancing agents, stability enhancing agents, bioadhesive agents, controlled release agents, flow aids and processing aids. More particularly, suitable excipients include cellulose ethers, acrylic acid polymers, and bile salts. Other suitable excipients are described in detail in the Handbook of Pharmaceutical Excipients, published jointly by the American Pharmaceutical Association and The Pharmaceutical Society of Great Britain, the Pharmaceutical Press, 1986, relevant portions incorporated herein by reference. Such excipients are commercially available and/or can be prepared by techniques known in the art.

The excipients may also be chosen alone or in combination to modify the intended function of the effective ingredient by improving flow, or bio-availability, or to control or delay the release of the effective ingredient. Specific nonlimiting examples include: Span 80, Tween 80, Brij 35, Brij 98, Pluronic, sucroester 7, sucroester 11, sucroester 15, sodium lauryl sulfate, oleic acid, laureth-9, laureth-8, lauric acid, vitamin E TPGS, Gelucire 50/13, Gelucire 53/10, Labrafil, dipalmitoyl phosphadityl choline, glycolic acid and salts, deoxycholic acid and salts, sodium fusidate, cyclodextrins, polyethylene glycols, labrasol, polyvinyl alcohols, polyvinyl pyrrolidones and tyloxapol, cellulose derivatives, and polyethoxylated castor oil derivatives. Using the process of the present invention, the morphology of the effective ingredients can be modified, resulting in highly porous particles and respirable aggregates.

### Inhalers and Nebulizers

Delivery of the Voriconazole Inhalation Powder to the lung can be achieved through any suitable delivery means, including a nebulizer, a dry powder inhaler, or a metered dose inhaler. Those of ordinary skill in the art of pulmonary delivery will understand the details of operating such devices. More information about the operation of such devices can also be found in, for example, “The Mechanics of Inhaled Pharmaceutical Aerosols: An Introduction”, by W. Finlay, Academic Press, 2001; and in “Inhalation Aerosols” edited by A. J. Hickey, Marcel Dekker, New York, 1996, both of which are incorporated herein by reference.

In certain specific embodiments, a Plastiape Monodose RS00 high resistance device is used with Voriconazole Inhalation Powder including Plastiape RS01 high and medium resistance devices and an RS00 high resistance device. In certain embodiments, the RS00 high resistance device exhibited consistent performance as a function of pressure drop through the device. In other embodiments, the capsule-based DPI device (e.g., RS00 Model 8 Monodose [RS00-0601212V1-M.8] manufactured by Plastiape) is used for administration of Voriconazole Inhalation Powder.

One of ordinary skill will recognize that the performance of the DPI itself is a combination of characteristics of the drug, the capsule, and the DPI device itself. In certain embodiments, the DPI device uses incoming air to efficiently empty the capsule (e.g., a hard, two-piece Size 3 capsule) while the centrifugal spinning action maximizes aerosolization of the powder formulation. The device is small, lightweight, and designed as a high resistance device.

### Manufacturing Process Development

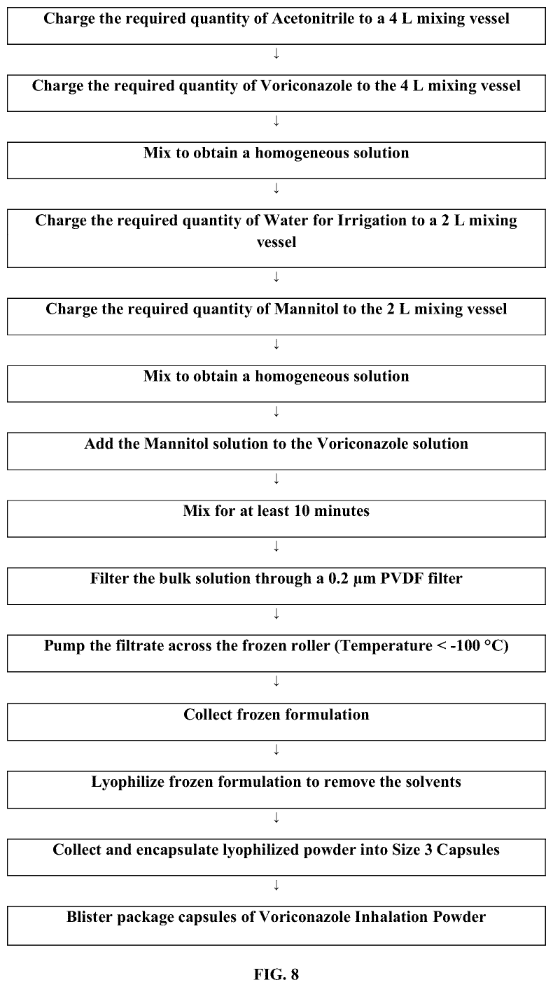

The thin film freezing (TFF) process involves rapidly freezing a solution of a drug substance and excipients on a cryogenically-cooled solid surface. This process has been shown to produce low density pharmaceutical powders composed of porous nanostructured aggregate particles. TFF-processed powders are highly respirable when aerosolized with a commercially marketed dry powder inhaler (e.g., Plastiape Monodose). The TFF process is designed to achieve a powder with a low density, nanostructured morphology. A flow diagram of the manufacturing process of the Voriconazole Inhalation Powder is illustrated in FIG. 8.

Voriconazole can be made using commercially available reagents and starting materials using, for example, the following synthetic procedure:

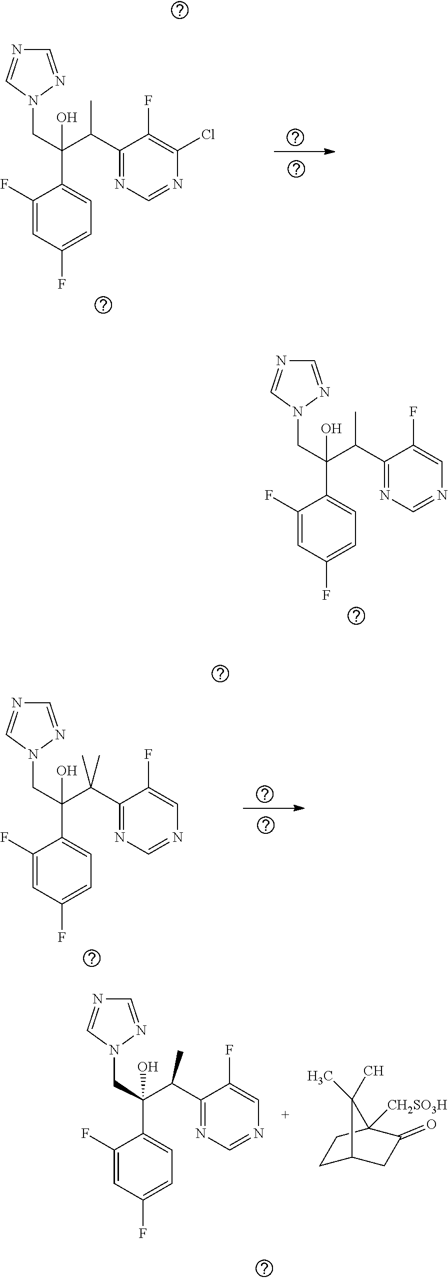

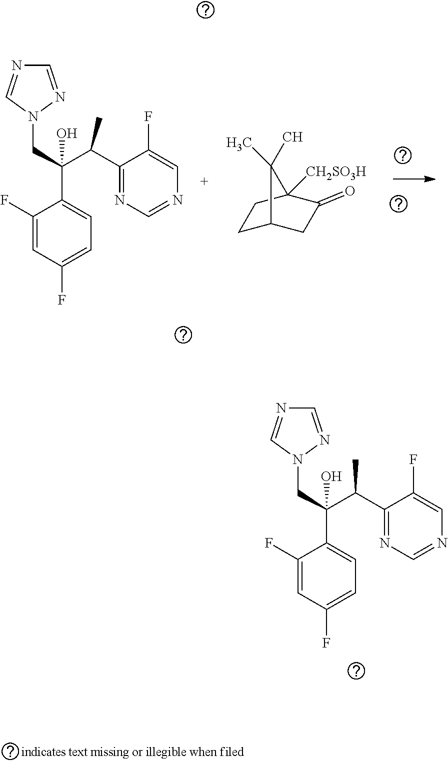

Voriconazole has two chiral centers, hence there are possible four stereo isomers (i.e., Voriconazole (I), its Enantiomer (II), and two Diastereomers III and IV (racemic mixture #2S,3S/2R,3R). These chemical structures are illustrated below.

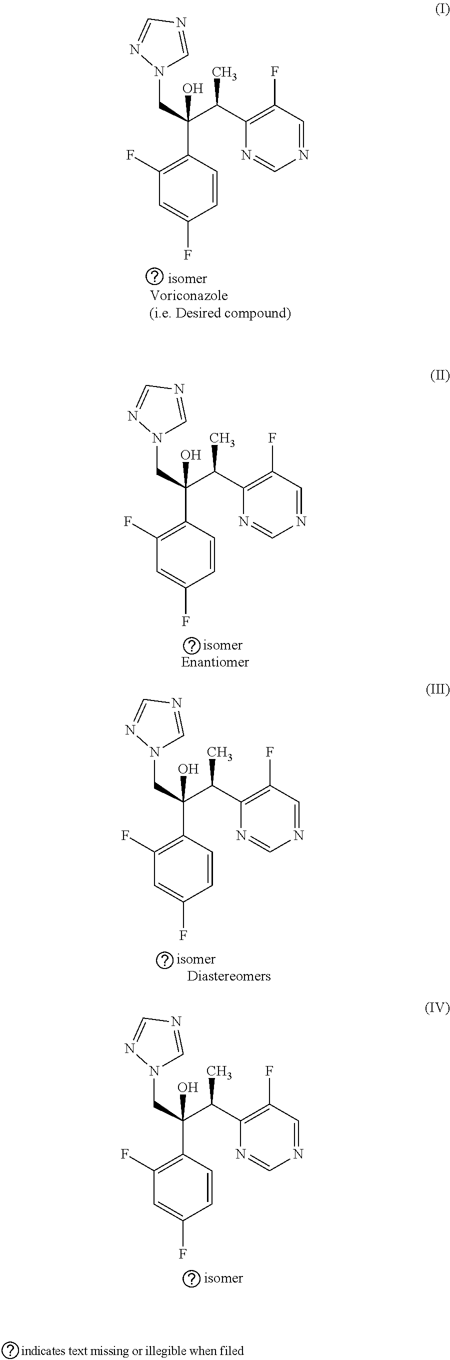 [SMILES: C[C@@H](c1ncncc1F)[C@](O)(Cn1cncn1)c1ccc(F)cc1F ; C[C@@H](c1ncncc1F)[C@](O)(Cn1cncn1)c1ccc(F)cc1F ; C[C@@H](c1ncncc1F)[C@](O)(Cn1cncn1)c1ccc(F)cc1F ; C[C@@H](c1ncncc1F)[C@](O)(Cn1cncn1)c1ccc(F)cc1F]

A high yielding diastereoselective process to form (2R,3S)-2-(2,4-difluro-phenyl)-3-(5-flouro-4-pyrimidinyl)-1 (1H-1,2,4-triazol-1-yl)2-butanol was developed. Enantiomer (II) is controlled in the finished product specification with a limit of NMT 0.13% by HPLC. The diastereomers (III) and (IV) are controlled in VOC1 (supra) intermediate specification as diastereomer impurity (i.e., racemic mixture 2S,3S/2R,3R) with NMT 0.5% of related substances by HPLC. It was determined that the Voriconazole is consistently the (2R,3S) isomer and remaining stereo isomers Enantiomer (II) and Diastereomers (III & IV) are controlled in the drug substance.

The solution concentration, freezing temperature and lyophilization cycle (including shelf temperature and hold time/ramp rate), are varied in a systematic way to identify critical process parameters. Voriconazole was dissolved in acetonitrile and mannitol was dissolved in water by continuous stirring. The organic and aqueous phases were combined with continuous stirring to form a co-solvent mixture. The final solutions (total volume 2.0-3.7 L) consisted of acetonitrile:water 50:50 (v:v) containing 10-30 mg/mL total solids. A summary is illustrated in Table 8.

TABLE 8
Summary of Development Voriconazole/Mannitol Batches (10-20 mg/mL Solids)

| Lot Number | 1 | 2 | 3 | 4 |
|---|---|---|---|---|
| Solvent System | acetonitrile:water 50:50 (v/v) | | | |
| Concentration (mg/mL) | 10 | 20 | 10 | 20 |
| Solution Volume (L) | 2.0 | 2.0 | 3.7 | 3.7 |
| Batch Size (g) | 20 | 40 | 37 | 75 |
| Drum Temperature (° C.) | −60 ± 10 | −150 ± 10 | −65 ± 10 | −150 ± 10 |
| Initial Cooling (min) | 50 | 110 | 55 | 120 |
| Flow Rate (mL/min) | 25 | 25 | 25 | 25 |
| Liquid N₂ Used (L) | ~100 | ~175 | ~150 | ~300 |
| Lyophilization | −40° C. hold 20 h | | −40° C. hold 2 h | −40° C. hold 2 h |
| (100 mTorr) | −40 to 25° C. ramp 3.25° C./h, 20 h | | −40 to 10° C. ramp | −40 to 25° C. ramp, |
| | 25° C. hold 20 h | | 10 h | 20 h |
| | | | −10° C. hold 18 h | 25° C. hold 42 h |
| | | | 15° C. hold 18 h | |
| | | | 25° C. hold 59 h | |
| Dry Yield (%) | 72.8 | 94.8 | 89.5 | 86.9 |

The solutions were then frozen by drop-wise addition via an 8-channel pump head from about a 10 cm height onto a rotating (e.g., 20 rpm) cryogenic steel surface (precooled with liquid nitrogen to about −150° C. to produce thin films. The frozen films were removed from the steel surface by a scraper and collected into pre-chilled stainless steel trays.

Once enough frozen materials (0.7-1.2 L) were collected to fill a tray, the tray was quickly transferred to a three-shelf lyophilizer with shelf temperature prechilled to about −55° C. A lyophilization cycle was started when all three trays were filled with frozen material (total 2-3.7 L) and placed into the lyophilizer. Solvents were sublimated over about 60-65 hours at ultimate chamber pressures of approximately 100 mTorr while the shelf temperature was gradually ramped from about −40° C. to about +25° C.

The dried powders were removed from the lyophilizer after ambient air was fed into the lyophilization chamber to equilibrate to atmospheric pressure, then stored at room temperature in double LDPE bag zip-tied with desiccant placed between bags.

The Voriconazole Inhalation Powder was filled into a capsule (e.g., Size 3 HPMC capsules) and aerosolized using Plastiape RS01 low resistance dry powder inhaler and tested for aerodynamic performance using a Next Generation Impactor (NGI).

### EXAMPLES

### Example 1

Pharmacokinetic analysis of Voriconazole Inhalation Powder following oral inhalation administered using the Plastiape Monodose RS00, high resistance inhaler device to healthy adults in a domiciled clinic setting to ensure continuous monitoring of subjects.

The first part comprised a SAD regimen in 4 escalating dose cohorts (Part 1) and, following demonstration of safety and tolerability in these subjects as assessed by the Medical Monitor and an independent Data Safety Monitoring Board (DSMB) at each dose level, Part 2 of the study commenced as a MAD regimen (BID for 13 doses delivered over 7 days) across 4 escalating dose cohorts. All cohorts in Part 1 (SAD) were completed sequentially and the Part 2 (MAD) dosing commenced following establishment of safety of the total daily administered dose in the SAD part (i.e., the dosing of 10 mg/kg BID in the MAD part commenced when 20 mg/kg/day in the SAD part was shown to be safe).

An independent and medically qualified DSMB provided oversight for the study and was responsible for review of unblinded safety data in order to make recommendations to the sponsor to escalate dosing, stop the study, or take additional measures to enhance subject safety.

Eligible healthy volunteers received single ascending doses (SAD—Part A) and multiple ascending doses (MAD—Part B) of Voriconazole Inhalation Powder taken BID for 7 days. The objective of the study was to evaluate the safety and PK profiles of escalating single doses of Voriconazole Inhalation Powder versus placebo. Safety, including physical examinations, laboratory tests, visual exams, and ECG measurements, were conducted throughout the course of dosing in both the SAD and MAD parts.

A total of 33 study subjects participated in Part A (SAD). Four cohorts of 8 study subjects (6 active drug, 2 placebo) were tested with doses escalated from 10 to 80 mg. Samples were collected for PK evaluations over 24 hours at each dose level in addition to the safety evaluations. Thirty-two subjects participated in Part B (MAD), with multiple dose administration of the same dose levels. Subjects received 13 total doses, administered BID (every 12 hours). The MAD portion of the study began after the safety results from the SAD Dose Level 2 were reviewed and progression was authorized by the Principal Investigator and TFF Medical Monitor. Pharmacokinetics samples were collected intensively after the first and last dose administrations.

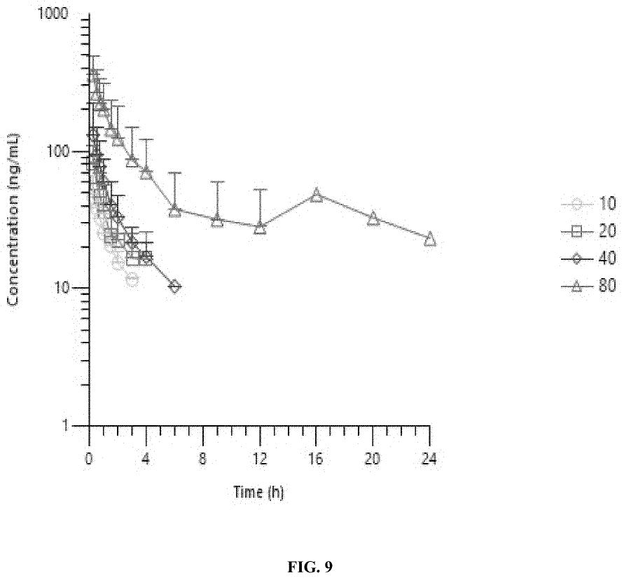

Voriconazole plasma concentrations were measured after each dose administration in all study subjects. FIG. 9 shows the mean concentrations±the standard deviation (SD) for each of the dose levels, with the PK parameters given from each dose level given in Table 9.

TABLE 9
Mean (±SD) of PK Parameters, All Treatments, SAD

| | | | | | HL | |
|---|---|---|---|---|---|---|
| Dose level, | Tₘₐₓ^{a} | Cₘₐₓ | AUCₗₐₛₜ | Tₗₐₛₜ^{a} | Lambda z | AUC₍₀₋₁₂₎ |
| mg | (h) | (ng/mL) | (h*ng/mL) | (h) | (h) | (h*ng/mL) |
| 10 ^{b} | 0.25 | 53.1 ± 12.0 | 56.6 ± 23.8 | 2.0 | 1.13 ± 0.434 | 76.6 ± 31.0 |
| 20 | 0.25 | 84.2 ± 30.1 | 95.6 ± 55.9 | 3.0 | 1.42 ± 0.656 | 124 ± 75.7 |
| 40 | 0.25 | 131 ± 89.6 | 176 ± 96.3 | 4.0 | 1.62 ± 0.416 | 203 ± 101 |
| 80 | 0.25 | 365 ± 124 | 889 ± 759 | 10.5 | 3.88 ± 2.34 | 828 ± 551 |

AUC₍₀₋₁₂₎ = area under the concentration-time curve from 0 to 12 hours; AUCₗₐₛₜ = area under the concentration-time curve to last measured concentration; Cₘₐₓ = maximum achieved concentration; CYP = cytochrome P; HL Lambda z = terminal half-life; h = hours; SD = standard deviation; Tₗₐₛₜ = time of last measurable concentration; Tₘₐₓ = time of maximum achieved concentration.
^{a}Median;
^{b}N = 5 for this group.

These data show that lung delivery of voriconazole results in very rapid absorption, with maximum concentrations (Cₘₐₓ) occurring shortly after inhalation for all dose levels. Plasma concentrations are relatively low compared to IV and oral administrations, likely a factor of the low doses given. Voriconazole concentrations are well below those associated with voriconazole-associated AEs.

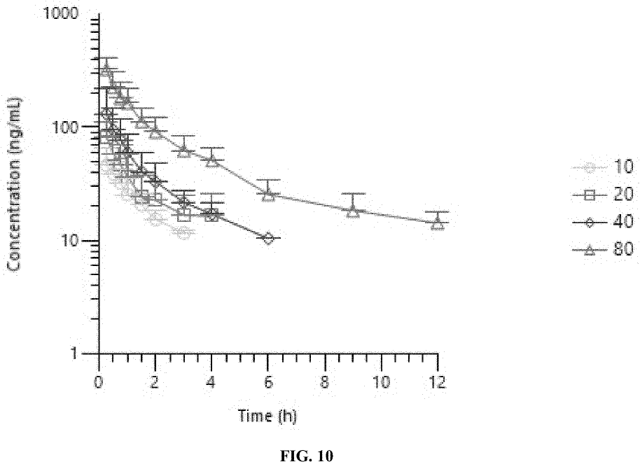

Voriconazole is primarily metabolized by CYP2C19, and voriconazole's disposition is known to be influenced by the genetic expression of this enzyme. Allelic profiling was performed for CYP2C19 in all study subjects, with 1 poor metabolizer identified enrolled in the 80 mg group. Table 10 and FIG. 10 provide a summary of the disposition from these administrations with the exclusion of the poor metabolizer.

TABLE 10
Mean (±SD) of PK Parameters, All Treatments, SAD: CYP2C19
Extensive Metabolizers Only

| Dose level, | Tₘₐₓ^{a} | Cₘₐₓ | AUCₗₐₛₜ | Tₗₐₛₜ^{a} | half-life | AUC₍₀₋₁₂₎ |
|---|---|---|---|---|---|---|
| mg | (h) | (ng/mL) | (h*ng/mL) | (h) | (h) | (h*ng/mL) |
| 10 ^{b} | 0.25 | 53.1 ± 12.0 | 56.6 ± 23.8 | 2.0 | 1.13 ± 0.434 | 76.6 ± 31.0 |
| 20 | 0.25 | 84.2 ± 30.1 | 95.6 ± 55.9 | 3.0 | 1.42 ± 0.656 | 124 ± 75.7 |
| 40 | 0.25 | 131 ± 89.6 | 176 ± 96.3 | 4.0 | 1.62 ± 0.416 | 203 ± 101 |
| 80 ^{b} | 0.25 | 325 ± 86.2 | 592 ± 223 | 9.02 | 3.15 ± 1.68 | 615 ± 202 |

AUC₍₀₋₁₂₎ = area under the concentration-time curve from 0 to 12 hours; AUCₗₐₛₜ = area under the concentration-time curve to last measured concentration; Cₘₐₓ = maximum achieved concentration; CYP = cytochrome P; SAD = single ascending dose; Tₗₐₛₜ = time of last measurable concentration; Tₘₐₓ = time of maximum achieved concentration.
^{a}Median.
^{b} N = 5 for this group.

Voriconazole is also known to exhibit nonlinear PK. Nonlinearity appears to be both dose-related and time-related. However, the available literature does not provide PK at doses as low as those administered in this study to determine the dose associated with nonlinearity. Review of the PK results from extensive metabolizers only suggest linearity persists through the 40 mg dose, and shows nonlinearity above 40 mg.

Intensive sample collections were performed after the first and last doses from the BID (every 12 hours) administrations of voriconazole. The mean (±SD) measurements from both Dose 1 and Dose 13 are provided in FIG. 11.

Two subjects in the 20 mg cohort were dropped from the study, 1 due to withdrawal of consent and the other due to an AE. In total, 4 of the 6 subjects randomized to active drug provided PK results summarized here in the 20 mg BID group.

A summary of the PK parameters estimated from the first and last doses (Doses 1 and 13, respectively) is provided in Table 11. The half-life was quite short for the 10, 20, and 40 mg cohorts, suggesting no accumulation would be present. The Cₘₐₓ and area under the concentration-time curve from 0 to 12 hours (AUC₍₀₋₁₂₎) values support that premise. As noted from the SAD, voriconazole disposition appears linear up to and including 40 mg; Dose 1 and Dose 13 PK summaries agree with that.

Dosing above 40 mg (e.g., 80 mg) suggests the introduction of nonlinearity. The PK exposure at Dose 1 shows a less than proportional increase when compared to the other doses. However, continued dosing shows a prolongation of voriconazole's half-life and a greater than proportional increase in the overall exposure relative to the other doses after Dose 13. This observation is consistent with what is known of voriconazole PK after oral and IV administrations.

TABLE 11
Mean (±SD) of PK Parameters, All Treatments, MAD, Doses 1 and 13, All Subjects

| | Dose | | | | | | | |
|---|---|---|---|---|---|---|---|---|
| Dose | level, | Tₘₐₓ^{a} | Cₘₐₓ | AUCₗₐₛₜ | Tₗₐₛₜ^{a} | Half-life | AUC₍₀₋₁₂₎ | Cₘᵢₙ |
| no. | mg | (h) | (ng/mL) | (h*ng/mL) | (h) | (h) | (h*ng/mL) | (ng/mL) |
| 1 | 10 ^{b} | 0.25 | 32.7 ± 7.77 | 36.3 ± 9.23 | 2.0 | 1.28 ± 0.382c | 56.2 ± 15.4 | — |
| | 20^{d} | 0.49 | 44.7 ± 14.3 | 72.7 ± 31.1 | 2.76 | 1.47 ± 0.387 | 98.0 ± 29.8 | — |
| | 40 | 0.25 | 117 ± 45.3 | 233 ± 116 | 5.0 | 1.98 ± 0.559 | 261 ± 114 | — |
| | 80 ^{b} | 0.265 | 140 ± 64.4 | 275 ± 157 | 6.0 | 2.62 ± 2.11 | 301 ± 151 | = |
| 13 | 10 | 0.25 | 40.5 ± 11.0 | 45.4 ± 13.8 | 2.0 | 1.74 ± 0.767 | 73.5 ± 19.7 | 1.67 ± 4.08 |
| | 20^{d} | 0.375 | 50.5 ± 31.5 | 108 ± 131 | 2.51 | 1.60 ± 0.764 | 129 ± 130 | 0.0 ± 0.0 |
| | 40 | 0.25 | 143 ± 49.0 | 321 ± 185 | 6.0 | 2.66 ± 1.20 | 352 ± 174 | 2.67 ± 6.53 |
| | 80 ^{b} | 0.25 | 283 ± 207 | 1500 ± 2430 | 11.9 | 5.56 ± 4.72 | 1150 ± 1550 | 43.6 ± 90.2 |

AUC₍₀₋₁₂₎ = area under the concentration-time curve from 0 to 12 hours; AUCₗₐₛₜ = area under the concentration-time curve to last measured concentration; Cₘₐₓ = maximum plasma concentration; Cₘᵢₙ = minimum plasma concentration; CYP = cytochrome P; HL Lambda z = terminal half-life; MAD = multiple ascending dose; PK = pharmacokinetic; SD = standard deviation; Tₗₐₛₜ = time of last measurable concentration; Tₘₐₓ = time of maximum achieved concentration.
^{a}Median.
^{b} N = 5 for this group.
^{c}N = 4.
^{d}N = 4 for this group.

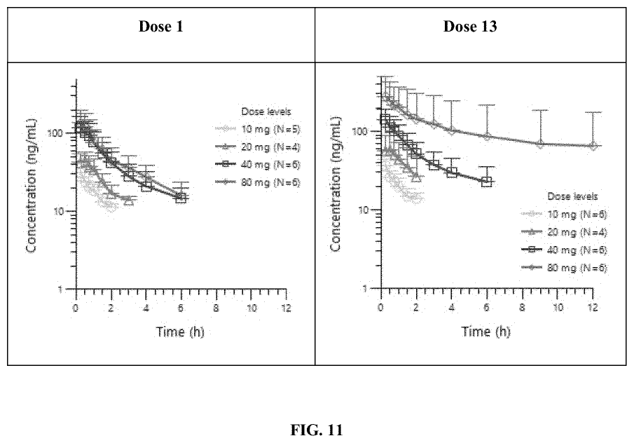

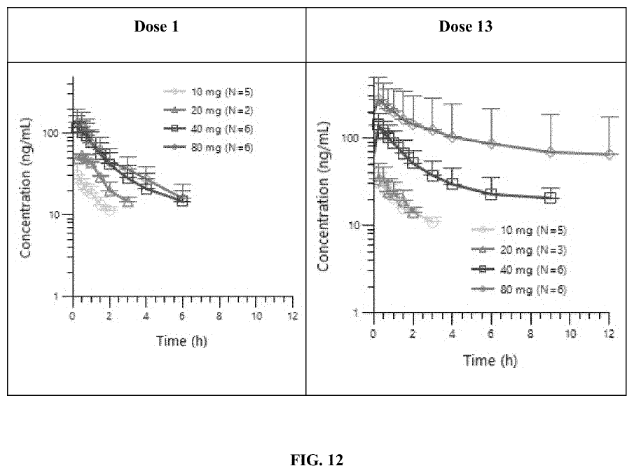

Two subjects enrolled in this portion of the study were identified as poor metabolizers. Both subjects were part of the 20 mg BID cohort. FIG. 12 shows the mean concentrations for only the extensive metabolizers. 2 subjects in the 20 mg BID group were identified as extensive metabolizers from Dose 1, and 3 subjects were identified as extensive metabolizers from Dose 13 (1 of the extensive metabolizers subjects had all voriconazole concentrations below quantifiable limits from Dose 1).

TABLE 12
Mean ± SD of PK Parameters, All Treatments, MAD, Doses 1 and 13:
Extensive Metabolizers Only

| | Dose | | | | | | | |
|---|---|---|---|---|---|---|---|---|
| Dose | level, | Tₘₐₓ^{a} | Cₘₐₓ | AUCₗₐₛₜ | Tₗₐₛₜ^{a} | Half-life | AUC₍₀₋₁₂₎ | Cₘᵢₙ |
| no. | mg | (h) | (ng/mL) | (h*ng/mL) | (h) | (h) | (h*ng/mL) | (ng/mL) |
| 1 | 10 ^{b} | 0.25 | 32.7 ± 7.77 | 36.3 ± 9.23 | 2.0 | 1.28 ± 0.382^{c} | 56.2 ± 15.4 | — |
| | 20^{d} | 0.49 | 54.4 ± 1.34 | 94.2 ± 21.0 | 3.26 | 1.38 ± 0.622 | 117 ± 27.8 | — |
| | 40 | 0.25 | 117 ± 45.3 | 233 ± 116 | 5.0 | 1.98 ± 0.559 | 261 ± 114 | — |
| | 80 ^{b} | 0.265 | 140 ± 64.4 | 275 ± 157 | 6.0 | 2.62 ± 2.11 | 301 ± 151 | = |
| 13 | 10 | 0.25 | 40.5 ± 11.0 | 45.4 ± 13.8 | 2.0 | 1.71 ± 0.767 | 73.5 ± 19.7 | 1.67 ± 4.08 |
| | 20^{e} | 0.25 | 36.2 ± 16.0 | 43.4 ± 29.4 | 2.0 | 1.22 ± 0.148 | 65.0 ± 25.1 | 0.0 ± 0.0 |
| | 40 | 0.25 | 143 ± 49.0 | 321 ± 185 | 6.0 | 2.66 ± 1.20 | 352 ± 174 | 2.67 ± 6.53 |
| | 80 ^{b} | 0.25 | 283 ± 207 | 1500 ± 2430 | 11.9 | 5.56 ± 4.72 | 1150 ± 1550 | 43.6 ± 90.2 |

AUC₍₀₋₁₂₎ = area under the concentration-time curve from 0 to 12 hours; AUCₗₐₛₜ = area under the concentration-time curve to last measured concentration; Cₘₐₓ = maximum plasma concentration; Cₘᵢₙ = minimum plasma concentration; CYP = cytochrome P; HL Lambda z = terminal half-life; SD = standard deviation; Tₗₐₛₜ = time of last measurable concentration; Tₘₐₓ = time of maximum achieved concentration.
^{a}Median.
^{b} N = 5 for this group.
^{c}N = 4.
^{d}N = 2 for this group.
^{e}N = 3 for this group.

### Example 2

An additional study conducted to assess the safety, tolerability, and PK of Voriconazole Inhalation Powder in adult subjects with asthma is ongoing with preliminary data included herein. The study involves 2 cohorts of 8 subjects each randomized in a 3:1 ratio (6 active and 2 placebo) receiving 7 doses (over 3.5 days) of either 40 mg or 80 mg Voriconazole Inhalation Powder or placebo.

Plasma PK was assessed from serial blood collections following Dose 1 (Day 1) and Dose 7 (Day 4). Study treatment stopping rules for individual subjects were included.

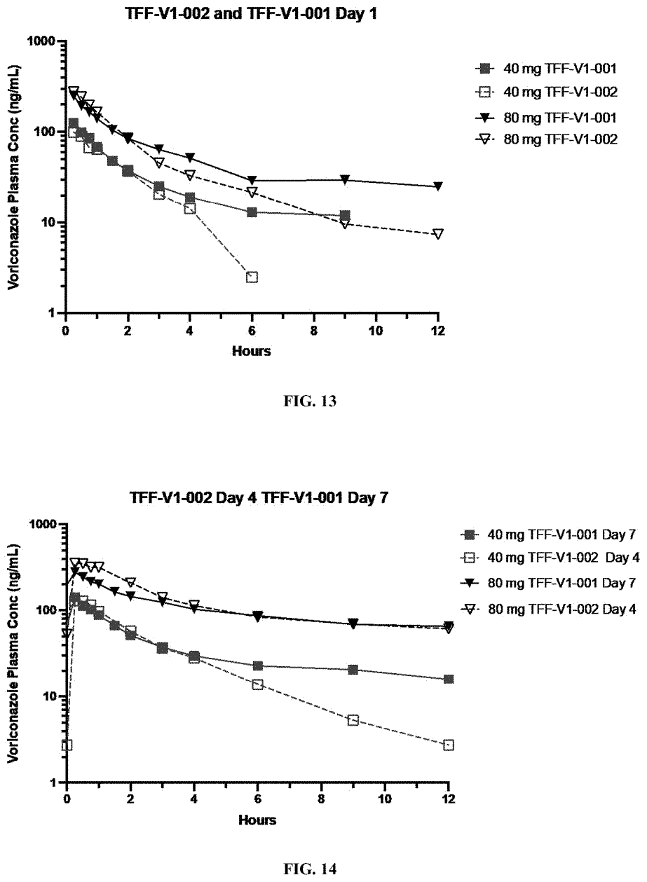

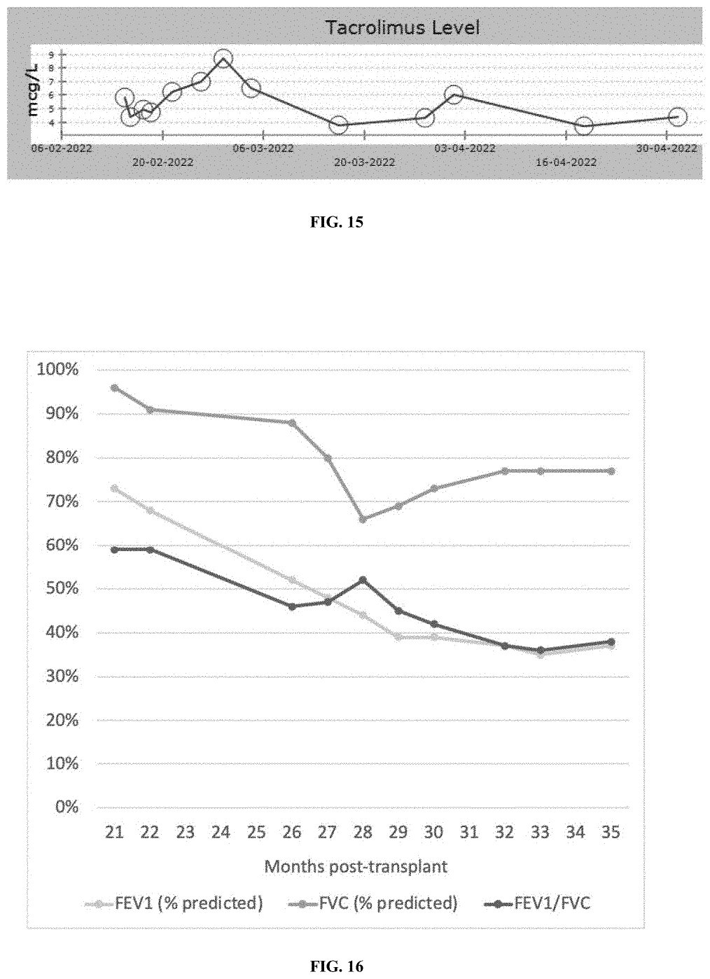

Averaged quality controlled concentration data from Day 1 and Day 4 from the individuals in Cohorts 1 and 2 (40 mg and 80 mg Voriconazole Inhalation Powder) were compared with the mean concentration time from the 40 mg and 80 mg cohorts from Example 1. The MAD and SAD cohorts for Example 1 were combined without separation of CYP2C19 poor metabolizers and extensive metabolizers and a comparison with Day 1 indicates a similar peak level and clearance of systemic exposure of voriconazole through 4-6 h between the subjects with reactive airways disease to healthy subjects from the 40 mg and 80 mg single dose administration (FIG. 13). The differences in observed mean concentrations at 6 h and later may be related to differences in CYP2C19 genetics between the 4 cohort populations. Note that the 6 h time point for the 40 mg TFF-V1-002 cohort is a single subject that was above the LLOQ while the other 5 were BLQ and assigned a value of 0 for generation of the mean.

Voriconazole mean systemic exposure for both 40 mg and 80 mg cohorts from both studies were plotted. Voriconazole Inhalation Powder was administered BID for 3.5 days (7 administrations) Example 1 and BID for 6.5 days (13 administrations) in Example 2. The mean systemic exposure was similar after repeat administration at both dose levels. The 80 mg cohort mean in Example 2 was slightly higher at 354 ng/mL compared to 281 ng/mL, but the standard deviation (not presented) clearly overlapped, suggesting no statistical difference (FIG. 14). The semilog plot for 3 of the 4 cohorts (excluding 40 mg Example 2) suggests similar absorption and clearance rates of voriconazole into systemic circulation for either a 40 mg or 80 mg dose after repeat administration between subjects with reactive airways disease and healthy subjects. The observed difference in Example 2, Day 4 40 mg cohort terminal phase clearance may be related to CYP genetics.

### Comparative Example 1

The PK of voriconazole presented here represents systemically administered voriconazole (e.g., oral or IV). Voriconazole PK has been characterized in healthy subjects, special populations and patients. Voriconazole demonstrates dose-dependent nonlinear behavior due to saturation of metabolism. There is a greater than proportional increase in exposure with an increase in dose. Accumulation results with multiple dose administrations. A summary of exposure after single and multiple dose administrations from both IV and oral administrations is given in Table 13.

TABLE 13
Voriconazole Exposure^{a} After Single and Multiple Doses, IV and Oral
Administrations, Different Dosing Regimens^{b}

| | | | | 400 mg | | |
|---|---|---|---|---|---|---|
| | 6 mg/kg IV | | | Oral | 200 mg | 300 mg |
| | (loading | 3 mg/kg | 4 mg/kg | (loading | Oral | Oral |
| | dose) | IV q12h | IV q12h | dose) | q12h | q12h |
| N | 35 | 23 | 450 | 17 | 48 | 16 |
| AUC₁₂ (μg:h/mL) | 13.9 (32) | 13.7 (53) | 33.9 (54) | 9.31 (38) | 12.4 (78) | 34.0 (53) |
| Cₘₐₓ (μg/mL) | 3.13 (20) | 3.03 (25) | 4.77 (36) | 2.30 (19) | 2.31 (48) | 4.74 (35) |
| Cₘᵢₙ (μg/mL) | — | 0.46 (97) | 1.73 (74) | — | 0.46 (120) | 1.63 (79) |

AUC₁₂ = area under the curve over 12-hour dosing interval; Cₘₐₓ = maximum achieved concentration, Cₘᵢₙ = minimum plasma concentration; CV = coefficient of variation; PK = pharmacokinetic.
^{a}Geometric mean (% CV)
^{b}from VFEND Package Insert.
Note:
Parameters were estimated based on non-compartmental analysis from 5 PK studies.

Based on a population PK analysis of pooled data in healthy subjects (N=207), the oral bioavailability of voriconazole is estimated to be 96% (coefficient of variation, CV 13%). Bioequivalence was established between the 200 mg tablet and the 40 mg/mL oral suspension.

Maximum plasma concentrations (Cₘₐₓ) are achieved 1-2 hours after dosing. When multiple doses of voriconazole are administered with high-fat meals, the mean Cₘₐₓ and AUC′ are reduced by 34% and 24%, respectively when administered as a tablet and by 58% and 37%, respectively, when administered as the oral suspension.

In healthy subjects, the absorption of voriconazole is not affected by coadministration of oral ranitidine, cimetidine, or omeprazole, drugs that are known to increase gastric pH.

Distribution from Oral or IV Administrations

The volume of distribution at steady state for voriconazole is estimated to be 4.6 L/kg, suggesting extensive distribution into tissues. Plasma protein binding is estimated to be 58% and was shown to be independent of plasma concentrations achieved following single and multiple oral doses of 200 mg or 300 mg (approximate range: 0.9-15 μg/mL). Varying degrees of hepatic and renal impairment do not affect the protein binding of voriconazole.

Voriconazole concentrations have been measured in both plasma and bronchoalveolar lavage (BAL) samples after 3 days of treatment (6 mg/kg q12 h IV ×2 doses, then 4 mg/kg IV q12h). Measurements are conducted in epithelial lining fluid (ELF) and alveolar macrophages (AM) at 4, 8, and 12 hours after the final voriconazole dose. Results, shown in Table 14 indicate substantially higher concentrations in BAL and AM when compared to plasma, well above the Aspergillus spp. MIC₉₀.

TABLE 14
Mean (SD) Steady-state Voriconazole Plasma,
ELF, and AM Concentrations during Voriconazole
4 mg/kg q12h IV Therapy

| Time of | Plasma, | ELF, | AMs, |
|---|---|---|---|
| BAL (h) | μg/mL | μg/mL | μg/g |
| 4 | 5.3 (1.4) | 48.3 (7.6) | 20.6 (4.5) |
| 8 | 1.7 (0.9) | 10.1 (10.8) | 10.3 (8.5) |
| 12 | 2.2 (1.1) | 17.2 (13.3) | 14.4 (13.1) |

AM = alveolar macrophages; BAL = bronchoalveolar lavage; ELF = epithelial lining fluid; SD = standard deviation. Values are estimates based upon the results of five participants per collection time.

Another study reviewed the use of prophylactic voriconazole use in lung transplant patients and determined matched plasma and ELF concentrations at 2, 4, or 8 weeks post-transplant. The patients had received 6 mg/kg IV q12 h ×2 doses, then 200 mg orally BID for approximately 4 months. A total of 12 lung transplant patients were included in this assessment, with results given in Table 15. In all cases, the voriconazole concentrations in the lung ELF exceeded those of the plasma, with a mean (SD) ELF:plasma ratio of 11.

TABLE 15
Plasma and ELF Voriconazole Concentrations Receiving
Prophylactic Treatment (200 mg p.o. BID) Determined in
Lung Transplant Patients at 2, 4, or 8 Weeks Post-transplant

| | | | Timing | | | |
|---|---|---|---|---|---|---|
| | | | of dose | | | |
| | | | prior to | Voriconazole | | |
| | Type of | No. of | broncho- | concentration | | ELF/ |
| | lung | oral | scopy | (μg/ml) | | plasma |
| Subject | transplant | doses | (h) | Plasma | ELF | ratio |
| 1 | Double | 164 | 0.5 | 0.19 | 1.98 | 11 |
| 2 | Single left | 27 | 3.0 | NA^{a} | 4.73 | NA |
| 3 | Double | 114 | 3.0 | 1.35 | 13.28 | 10 |
| 4 | Single left | 30 | 4.0 | 1.34 | 7.85 | 6 |
| 5 | Single right | 98 | 4.5 | 0.76 | 1.58 | 2 |
| 6 | Single left | 36 | 5.0 | 2.66 | 44.00 | 17 |
| 7 | Single left | 34 | 5.0 | 2.10 | 57.90 | 28 |
| 8 | Single left | 77 | 6.0 | 4.56 | 83.32 | 18 |
| 9 | Double | 38 | 6.5 | 0.05 | 0.29 | 6 |
| 10 | Single left | 27^{b} | 6.5 | 1.16 | 13.27 | 11 |
| 11 | Single left | 47 | 12.0 | 0.15 | 0.73 | 5 |
| 12 | Single left | 99 | 13.5 | 0.43 | 2.16 | 5 |
| Mean | | 66 | | | | 11 (8) |
| (SD) | | (44) | | | | |

### Comparative Example 2¹: From Inhalation of VFEND IV Solution when Nebulized

Voriconazole has been administered by inhalation (e.g., nebulization) with concentrations determined in plasma and BAL. In a small study, voriconazole 40 mg was inhaled BID for 2 days with comparisons made to oral voriconazole 400 mg BID on Day 1 and 200 mg BID on Day 2. Blood collections were made at 15, 30, and 60 minutes after dosing on Day 1, with BAL and blood samples collected 12 hours after the last dose. Voriconazole was measured in ELF and plasma from these collections. The concentrations determined from the determinations in ELF and Day 2 plasma are given in Table 16. The concentrations from the 40 mg inhalation were much lower, as expected, in both the ELF and plasma. However, the ELF:plasma ratio was higher for the inhalation administration (median ratio=21) when compared to the oral route (median ratio=8). The ELF:plasma ratio after oral administration is consistent with the results presented in Table 16.

TABLE 16
Voriconazole Concentrations^{a} in ELF and Plasma from
the First and Last Administration of Both Oral and
Inhalation (IV VFEND via nebulizer) Doses Given BID

| | | | Concentration (μg/mL) | |
|---|---|---|---|---|
| | | | 12 hours after last dose | |
| Route | N | Dose, mg BID | ELF | Plasma |
| Oral | 6 | 400 (Day 1), 200 | 8.83 | 1.224 |
| | | (Day 2) | (4.37-35.17) | (0.535-2.34) |
| Inhalation | 6 | 40 | 0.190 | 0.008 |
| | | | (0.055-0.318) | (0.004-0.026) |

BID = twice daily; CI = confidence interval; ELF = epithelial lining fluid.
^{a}Median (95% CI)

Metabolism

In vitro studies showed that voriconazole is metabolized by the human hepatic CP450 enzymes, CYP2C19, CYP2C9, and CYP3A4. In vivo studies indicate that CYP2C19 is significantly involved in the metabolism of voriconazole. This enzyme exhibits genetic polymorphism and can add to the variability of the metabolism and PK. Allelic polymorphisms of the wild type CYP2C19*1 (mainly CYP2C19*2, CYP2C19*3 [inactive alleles] and CYP2C19*17 [ultra-active allele]) exist for CYP2C19 expression, with approximately 3-5% of European and 15-20% of Asian populations being poor metabolizers with no CYP2C19 function (a diplotype expression of CYP2C19*2/*2, CYP2C19*2/*3, or CYP2C19*3/*3).

The major metabolite of voriconazole is the N-oxide, which accounts for 72% of the circulating radiolabeled metabolites in plasma. Since this metabolite has minimal antifungal activity, it does not contribute to the overall efficacy of voriconazole.

Excretion

Voriconazole is eliminated via hepatic metabolism with less than 2% of the dose excreted unchanged in the urine. After administration of a single radiolabeled dose of either oral or IV voriconazole, preceded by multiple oral or IV dosing, approximately 80% to 83% of the radioactivity is recovered in the urine. The majority (>94%) of the total radioactivity is excreted in the first 96 hours after both oral and IV dosing.

As a result of non-linear PK, the terminal half-life of voriconazole is dose dependent and therefore not useful in predicting the accumulation or elimination of voriconazole.

Drug Interactions: Effect of Other Drugs Upon Voriconazole Pharmacokinetics

Voriconazole is metabolized by the human hepatic cytochrome P450 enzymes CYP2C19, CYP2C9, and CYP3A4. Results of in vitro metabolism studies indicate that the affinity of voriconazole is highest for CYP2C19, followed by CYP2C9, and is appreciably lower for CYP3A4. Inhibitors or inducers of these 3 enzymes may increase or decrease voriconazole systemic exposure (plasma concentrations), respectively.

Significant reductions in voriconazole concentrations can be expected when coadministered with rifampin, ritonavir, St. John's Wort, carbamazepine, or long-acting barbiturates. All of these drugs are considered contraindicated with voriconazole.

Drug Interactions: Effect of Voriconazole Upon the Pharmacokinetics of Other Drugs

Voriconazole, an azole antifungal compound, is in the group of drugs known to be strong CYP3A inhibitors. Voriconazole is no exception. In vitro studies show that voriconazole inhibits the metabolic activity of CYP2C19, CYP2C9, and CYP3A4. In vitro studies also show that the major metabolite of voriconazole, voriconazole N-oxide, inhibits the metabolic activity of CYP2C9 and CYP3A4. Thus, voriconazole has the potential to have significant interactions with other drugs.

Voriconazole is contraindicated with the following CYP3A4 substrates, because it can increase their concentrations to potentially dangerous values: sirolimus, terfenidine, atemizole, cisapride, pimozide, quinidine, everolimus, and ergot alkaloids.

Voriconazole is expected to increase the exposure of the following drugs, and downward dose adjustments may be necessary: alfentanyl, fentanyl, oxycodone, methadone, cyclosporine, tacrolimus, warfarin/coumarin, statin drugs, benzodiazepines, calcium channel blockers (nifendipine, felodipine, nicardipine, etc.), sulfonylureas (tolbutamide, glipizide, glyburide, etc.), vinca alkaloids, omeprazole, and some non-steroidal anti-inflammatory drugs (NSAIDS, ibuprofen, diclofenac).

Two-Way Interactions

Some interactions can affect both voriconazole as well as another coadministered drug.

Voriconazole is contraindicated with rifabutin (a CYP3A4 inducer and substrate) and efavirenz (a CYP3A4 inhibitor/inducer and substrate), based upon the effects of each drug upon one another.

Phenytoin, a CYP2C9 substrate and CYP inducer, would require frequent monitoring of both phenytoin and voriconazole. Lower doses than normal of phenytoin and higher doses than normal of voriconazole may be required. When oral contraceptives and voriconazole are given together, each can increase the concentrations of the other. Monitoring for AEs related to either oral contraceptives or voriconazole is recommended.

Pharmacodynamics in Humans

The primary PD endpoint with voriconazole is the resolution of the infection. Voriconazole use can also be related to some safety factors, including hepatotoxicity and neurotoxicity.

The VFEND product information states that a PK/PD analysis of patient data from 6 of 10 clinical trials (N=280) could not detect a positive association between mean, maximum, or minimum plasma voriconazole concentration and efficacy. However, PK/PD analyses of the data from all 10 clinical trials (N=1121) identified positive associations between plasma voriconazole concentrations and rate of both liver function test abnormalities and visual disturbances.

Relationship of Voriconazole Concentrations to Antifungal Efficacy and Toxicity

A meta-analysis was conducted across 21 studies involving 1158 patients treated with either IV or oral therapy. Trough plasma voriconazole concentrations were determined in these patients and evaluated for antifungal efficacy and toxicity. The analysis found that trough concentrations >0.5 μg/mL resulted in significantly greater antifungal efficacy, although review of the results suggested >1.0 μg/mL may be a better indicator. An increased risk of hepatotoxicity was found with trough voriconazole concentrations >3.0 μg/mL, and concentrations >4.0 μg/mL were associated with an increased risk of neurotoxicity. Other publications suggest that plasma concentrations of 1.0-2.0 μg/mL are needed to ensure higher success rates, but these studies were based upon fewer patients. Therapeutic drug monitoring is the standard of care with the use of systemic voriconazole, and will be planned for Phase 2 and Phase 3 studies of inhaled voriconazole.

### Example 3: Pharmacokinetic/Pharmacodynamic Summary

The PK of Voriconazole Inhalation Powder has been summarized from the completed Phase 1 study discussed in Example 1. Oral and IV voriconazole PK have been summarized from various literature sources and the VFEND prescribing information. Twice daily dosing is appropriate for drug administration by the oral and IV routes and is expected for the inhalation route.

Measured lung ELF voriconazole concentrations after IV or oral administration are 8 to 11 times greater (on average) than plasma concentrations collected at the same time. Small studies conducted with nebulized voriconazole for inhalation have shown efficacy at lower doses than IV or oral administration. Nebulized voriconazole provides a higher ELF:plasma ratio when compared to IV or oral voriconazole, suggesting a possibility for lower doses with targeted delivery for lung fungal infections. These lower doses may provide greater safety for voriconazole, with, potentially, a reduced chance to achieve plasma voriconazole concentrations associated with liver and neurotoxicity.

Therapeutic drug monitoring can reduce the incidence of AEs related to voriconazole. CYP2C19 genetic screening can also enhance optimal antifungal therapy.

All references cited herein, including patents, patent applications, papers, text books, and the like, and the references cited therein, to the extent that they are not already, are hereby incorporated herein by reference in their entirety. Comparative Examples 1 and 2 were compiled based on publicly available information.

The foregoing written specification is considered to be sufficient to enable one skilled in the art to practice the invention. The foregoing description and examples detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof

What is claimed is:

1. A method of treating a fungal infection in a subject in need thereof, the method comprising administering by inhalation to said subject voriconazole or a salt thereof in an amount of about 20 mg to about 80 mg, wherein the administration by inhalation achieves a maximum circulating plasma concentration of voriconazole of less than 1,000 ng/mL.

2. The method of claim 1, wherein the maximum circulating plasma concentration of voriconazole is less than 500 ng/mL.

3. The method of claim 1, wherein the maximum circulating plasma concentration of voriconazole is less than 300 ng/mL.

4. The method of claim 1, wherein the fungal infection is caused by Aspergillus fumigatus, Aspergillus flavus, Aspergillus niger, Aspergillus terreus, Candida albicans, Candida glabrata, Candida krusei, Candida parapsilosis, Candida tropicalis, Fusarium spp., Fusarium solani, Scedosporium apiospermum, Candida lusitaniae, or Candida guilliermondii.

5. The method of claim 1, wherein the fungal infection is invasive Aspergillosis, Candidemia, or esophageal Candidiasis.

6. The method of claim 1, wherein the subject is suffering from a reactive airway disorder.

7. The method of claim 6, wherein the reactive airway disorder is chronic obstructive pulmonary disease (COPD).

8. The method of claim 1, wherein the subject is immunodeficient.

9. The method of claim 8, wherein the immunodeficiency is caused by factors comprising neutropenia, hematopoietic stem cell transplant (HSCT), solid organ transplantation, prolonged therapy with high-dose corticosteroids, hematological malignancy, cytotoxic therapy, advanced acquired immune deficiency syndrome (AIDS), chronic granulomatous disease, and patients with liver failure.

10. The method of claim 1, wherein the administration is at least once per day.

11. The method of claim 1, wherein the administration is at least twice per day.

12. The method of claim 1, wherein the administration of voriconazole or a salt thereof comprises an amount of about 40 mg to about 120 mg.

13. The method of claim 1, wherein the administration of voriconazole or a salt thereof comprises an amount of about 60 mg to about 80 mg.

14. The method of claim 1, wherein the administration of voriconazole or a salt thereof comprises an amount of about 80 mg.

15. The method of claim 1, wherein the voriconazole or a salt thereof is voriconazole inhalation powder.

16. A method of increasing delivery of voriconazole to the lung of a subject in need thereof, the method comprising administering to said subject by inhalation voriconazole or a salt thereof in an amount of about 20 mg to about 80 mg, wherein the delivery of voriconazole to the lung by inhalation results in about 50-fold higher relative concentration in the lung compared to the oral administration of voriconazole after about 12 hours following administration.

17. The method of claim 16, the delivery of voriconazole to the lung results in about 100-fold higher relative concentration in the lung compared to the oral administration of voriconazole.

18. The method of claim 16, the delivery of voriconazole to the lung results in about 200-fold higher relative concentration in the lung compared to the oral administration of voriconazole.

19. The method of claim 16, wherein the subject is suffering from a fungal infection caused by Aspergillus fumigatus, Aspergillus flavus, Aspergillus niger, Aspergillus terreus, Candida albicans, Candida glabrata, Candida krusei, Candida parapsilosis, Candida tropicalis, Fusarium spp., Fusarium solani, Scedosporium apiospermum, Candida lusitaniae, or Candida guilliermondii.

20. The method of claim 12, wherein the subject is suffering from a fungal infection caused by Invasive Aspergillosis, Candidemia, or esophageal Candidiasis.

21. The method of claim 16, wherein the subject is immunodeficient.

22. The method of claim 21, wherein the immunodeficiency is caused by factors comprising neutropenia, hematopoietic stem cell transplant (HSCT), solid organ transplantation, prolonged therapy with high-dose corticosteroids, hematological malignancy, cytotoxic therapy, advanced acquired immune deficiency syndrome (AIDS), chronic granulomatous disease, and patients with liver failure.

23. The method of claim 16, wherein the administration is at least once per day.

24. The method of claim 16, wherein the administration is at least twice per day.

25. The method of claim 16, wherein the administration of voriconazole or a salt thereof comprises an amount of about 40 mg to about 80 mg.

26. The method of claim 16, wherein the administration of voriconazole or a salt thereof comprises an amount of about 60 mg to about 120 mg.

27. The method of claim 16, wherein the administration of voriconazole or a salt thereof comprises an amount of about 80 mg.

28. The method of claim 16, wherein the voriconazole or a salt thereof is voriconazole inhalation powder.

29. A method for reducing systemic circulation of voriconazole compared with the oral administration of voriconazole comprising administering to said subject by inhalation voriconazole or a salt thereof in an amount of about 20 mg to about 80 mg, wherein the administration by inhalation achieves a maximum circulating plasma concentration of voriconazole of less than 1,000 ng/mL.

30. The method of claim 29, wherein the maximum circulating plasma concentration of voriconazole is less than 500 ng/mL.

31. The method of claim 29, wherein the maximum circulating plasma concentration of voriconazole is less than 300 ng/mL.

32. The method of claim 29, wherein the fungal infection is caused by Aspergillus fumigatus, Aspergillus flavus, Aspergillus niger, Aspergillus terreus, Candida albicans, Candida glabrata, Candida krusei, Candida parapsilosis, Candida tropicalis, Fusarium spp., Fusarium solani, Scedosporium apiospermum, Candida lusitaniae, or Candida guilliermondii.

33. The method of claim 29, wherein the fungal infection is Invasive Aspergillosis, Candidemia, or esophageal Candidiasis.

34. The method of claim 29, wherein the subject is immunodeficient.

35. The method of claim 34, wherein the immunodeficiency is caused by factors comprising neutropenia, hematopoietic stem cell transplant (HSCT), solid organ transplantation, prolonged therapy with high-dose corticosteroids, hematological malignancy, cytotoxic therapy, advanced acquired immune deficiency syndrome (AIDS), chronic granulomatous disease, and patients with liver failure.

36. The method of claim 29, wherein the administration is at least once per day.

37. The method of claim 29, wherein the administration is at least twice per day.

38. The method of claim 29, wherein the administration of voriconazole or a salt thereof comprises an amount of about 40 mg to about 120 mg.

39. The method of claim 29, wherein the administration of voriconazole or a salt thereof comprises an amount of about 60 mg to about 80 mg.

40. The method of claim 29, wherein the administration of voriconazole or a salt thereof comprises an amount of about 80 mg.

41. The method of claim 1, wherein the voriconazole or a salt thereof is voriconazole inhalation powder.
